(19) United States
(12) Patent Application Publication
(10) Pub. No.: US 2004/0241776 A1
(43) Pub. Date: Dec. 2, 2004

(54) Multiplex enzyme-linked immunosorbent assay for detecting multiple analytes

(75) Inventors: Rebecca L. Geister, Cassopolis, MI (US); Murali D. Bandla, Granger, IN (US); Chester L. Sutula, Elkhart, IN (US)

Correspondence Address: MCLEOD & MOYNE, P.C., 2190 COMMONS PARKWAY, OKEMOS, MI, 48864, US

(73) Assignee: Agdia, Inc., Elkhart, IN

(21) Appl. No.: 10/843,807

(22) Filed: May 12, 2004

Related U.S. Application Data
(60) Provisional application No. 60/472,861, filed on May 22, 2003.

(51) Int. Cl. G01N 33/53; G01N 33/537; G01N 33/543

(52) U.S. Cl. 435/7.92

(57) ABSTRACT

A multiplex enzyme-linked immunosorbent assay (ELISA) using chromogenic substrates for detecting multiple analytes is described.

### CROSS-REFERENCE TO RELATED APPLICATION

This application claims priority to U.S. Provisional Patent Application 60/472,861, filed May 22, 2003.

### STATEMENT REGARDING FEDERALLY SPONSORED RESEARCH OR DEVELOPMENT

Not applicable.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to a multiplex enzyme-linked immunosorbent assay (ELISA) using chromogenic substrates for detecting multiple analytes.

(2) Description of Related Art

ELISA was developed by Engvall et al., Immunochem. 8: 871 (1971) and further refined by others such as Ljunggren et al. J. Immunol. Meth. 88: 104 (1987) and Kemeny et al., Immunol. Today 7: 67 (1986). ELISA and its variations are well known in the art.

In the prior art, a single ELISA is used to detect a single analyte or antibody using an enzyme-labeled antibody and a chromogenic substrate. To detect more than one analyte in a sample, a separate ELISA must be performed to detect each analyte. For example, to detect two analytes, two separate ELISA plates or two sets of wells are needed: a plate or set of wells for each analyte. Thus, prior art chromogenic-based ELISAs can detect only one analyte at a time. This a big limitation for detecting diseases with more than one marker or transgenic organisms which express more than one transgenic product.

Macri, J. N., et al., Ann Clin Biochem 29: 390-396 (1992)) describe an indirect assay wherein antibodies (Reagent-1) are reacted first with the analyte and then second labeled anti-antibodies (Reagent-2) are reacted with the antibodies. The result is a need for two separate washing steps which defeats the purpose of the direct assay. There is no suggestion that Reagents 1 and 2 could be combined in a mixture.

It would be an improvement over the prior art if there was a way to detect more than one analyte in chromogenic substrate-based ELISAs which used a single plate instead of separate plates to detect each analyte.

### SUMMARY OF THE INVENTION

The present invention provides a method for detecting two or more analyte species in a single enzyme-linked immunosorbent assay (ELISA), which comprises (a) providing for each analyte species to be detected, an antibody specific for the analyte species immobilized on a solid support; (b) contacting the antibodies immobilized on the solid support to a liquid sample suspected of containing at least one of the analyte species for a time sufficient for the antibodies to bind the analyte species; (c) removing the solid support from the liquid sample and washing the solid support to remove unbound material; (d) contacting the solid support to a solution comprising for each analyte species to be detected, an antibody specific for the analyte species to be detected conjugated to an enzyme label wherein the enzyme label for each antibody is different for a time sufficient for the antibodies to bind the analyte species bound by the immobilized antibodies; (e) removing the solid support from the solution and washing the solid support to remove unbound antibodies; and (f) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

The present invention further provides a method for detecting two or more analyte species in a single enzyme-linked immunosorbent assay (ELISA), which comprises (a) providing for each analyte species to be detected, an antibody specific for the analyte species immobilized on a solid support; (b) providing a solution comprising for each analyte species to be detected, an antibody specific for the analyte species to be detected conjugated to an enzyme label wherein the enzyme label for each antibody is different; (c) contacting a mixture comprising a sample suspected of containing at least one of the analyte species and the solution with the antibodies immobilized on the solid support for a time sufficient for the antibodies to bind the analyte species; (d) removing the solid support from the mixture and washing the solid support to remove unbound antibodies; and (e) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

The present invention further provides a method for detecting two analytes in a single enzyme-linked immunosorbent assay (ELISA), which comprises (a) providing a first antibody specific for a first analyte and a second antibody specific for a second analyte immobilized on a solid support; (b) contacting the antibodies immobilized on the solid support to a liquid sample suspected of containing one or both of the analytes for a time sufficient for the antibodies to bind the analytes; (c) removing the solid support from the liquid sample and washing the solid support to remove unbound material; (d) contacting the solid support to a solution comprising a third antibody specific for the first analyte and a fourth antibody specific for the second analyte wherein the third antibody is conjugated to a first enzyme label and the fourth antibody is conjugated to a second enzyme label for a time sufficient for the third and fourth antibodies to bind the analytes bound by the first and second antibodies; (e) removing the solid support from the solution and washing the solid support to remove unbound antibodies; (f) adding a first chromogenic substrate for the first enzyme label wherein conversion of the first chromogenic substrate to a detectable color by the first enzyme label indicates that the sample contains the first analyte; (g) removing the first chromogenic substrate; and (h) adding a second chromogenic substrate for the second enzyme label wherein conversion of the second chromogenic substrate to a detectable color by the second enzyme label indicates that the sample contains the second analyte.

In a further embodiment of the above methods, the solid support is a well of an ELISA plate.

In a further embodiment of the above methods, the chromogenic substrate is soluble and is converted to a soluble color. In further still embodiments, the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), PNPP or PNP (para-nitrophenyl phosphate), starch-iodine-penicillin V, bromothymol blue-penicillin V, and ONPG.

In a further still embodiment of the above methods, the enzyme label is selected from the group consisting of peroxidase, alkaline phosphatase, penicillinase, β-galactosidase, urease, and β-glucoronidase. In further still embodiments, the analytes are from plant pathogens or produced by transgenic plants.

The present invention further provides a method for determining whether a plant material is derived from a transgenic plant which comprises one or more heterologous genes by detecting the products produced by the one or more heterologous genes, which comprises (a) providing a liquid sample from the plant material; (b) providing a solid support having a mixture of antibodies immobilized thereon wherein the mixture comprises antibodies specific for the products produced by the one or more heterologous genes; (c) contacting the antibodies immobilized on the solid support to a liquid sample for a time sufficient for the antibodies to bind the products; (d) removing the solid support from the liquid sample and washing the solid support to remove unbound material; (e) contacting the solid support to a solution comprising for each product to be detected, an antibody specific for the product to be detected conjugated to an enzyme label wherein the enzyme label for each antibody is different for a time sufficient for the antibodies to bind the products bound by the immobilized antibodies; (f) removing the solid support from the solution and washing the solid support to remove unbound antibodies; and (g) determining whether the sample contains each product by sequentially detecting the enzyme labels by adding a chromogenic substrate specific for the enzyme label to be detected wherein conversion of the chromogenic substrate to a detectable color indicates the sample contains the product.

The present invention further provides a method for determining whether a plant material is derived from a transgenic plant which comprises one or more heterologous genes by detecting the products produced by the one or more heterologous genes, which comprises (a) providing a liquid sample from the plant material; (b) providing a solid support having a mixture of antibodies immobilized thereon wherein the mixture comprises antibodies specific for the products produced by the one or more heterologous genes; (c) providing a solution comprising for each product to be detected, an antibody specific for the product to be detected conjugated to an enzyme label wherein the enzyme label for each antibody is different for a time sufficient for the antibodies to bind the products bound by the immobilized antibodies to produce a mixture; (d) contacting the antibodies immobilized on the solid support to a mixture of the sample and solution for a time sufficient for the antibodies to bind the products; (e) removing the solid support from the mixture and washing the solid support to remove unbound material; and (f) determining whether the sample contains each product by sequentially detecting the enzyme labels by adding a chromogenic substrate specific for the enzyme label to be detected wherein conversion of the chromogenic substrate to a detectable color indicates the sample contains the product.

In a further embodiment of the above methods, the solid support is a well of an ELISA plate.

In a further embodiment of the above method, the chromogenic substrate is soluble and is converted to a soluble color. In further still embodiments, the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

In a further still embodiment of the above method, the enzyme label is selected from the group consisting of peroxidase, alkaline phosphatase, penicillinase, β-galactosidase, urease, and β-glucoronidase.

The present invention further provides a kit for an ELISA comprising (a) a microtiter plate having a multiplicity of wells, each well having immobilized therein a mixture of two or more antibody species wherein each antibody species is specific for a particular analyte species; (b) two or more first containers, each first container containing an antibody species conjugated to a particular enzyme label, wherein each antibody species is specific for the particular analyte species; and (c) two or more second containers, each second container containing a chromogenic substrate, wherein each chromogenic substrate is specific for the particular enzyme label.

In a further embodiment of the kit, the kit comprises one first container which contains a mixture of antibody species wherein each antibody species is specific for the particular analyte species and is conjugated to a particular enzyme label.

In a further embodiment of the kit, the chromogenic substrate is soluble and is converted to a soluble color.

In a further embodiment of the kit, the chromogenic substrate is selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

In a further embodiment of the kit, the enzyme label is selected from the group consisting of peroxidase, alkaline phosphatase, penicillinase, β-galactosidase, urease, and β-glucoronidase.

### OBJECTS

It is an object of the present invention to provide an ELISA for detecting a plurality of analytes in a single ELISA plate.

It is a further object of the present invention to provide an ELISA for detecting a plurality of analytes using chromogenic substrates.

It is a further object of the present invention to provide an ELISA for detecting transgenic plants which express more than one transgenic product.

These and other objects of the present invention will become increasingly apparent with reference to the following drawings and preferred embodiments.

### DESCRIPTION OF THE DRAWINGS

FIG. 1 shows a graph of the data in the table of File L which compares two-step with QTA using the PNP chromogenic substrate.

FIG. 2 shows a graph of the data in the table of File L which compares two-step ELISA with QTA using the TMB substrate.

FIG. 3 shows a chart of the multiplex ELISA response to 3Bb1 using anti-3Bb1 antibody-alkaline phosphatase conjugate and PNP chromogenic substrate.

FIG. 4 shows a chart of the multiplex ELISA response to 1Ab/1Ac (Cry1Ab/Cry1Ac) using anti-1Ab/1Ac antibody-peroxidase conjugate and TMB chromogenic substrate.

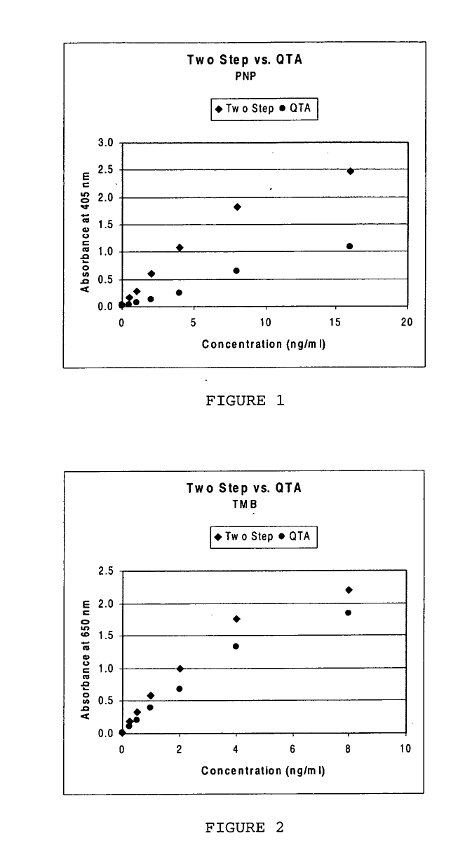

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The present invention relates to a method for detecting an antigen in a sample by means of an enzyme linked immunoassay (ELISA) using an enzyme labeled conjugate so that the enzyme label is detected in the assay by reaction with a chromogenic substrate for the enzyme, the improvement which comprises:

sequentially determining the presence of at least two different antigens in a single assay by two different enzymatic reactions of at least two enzyme labeled conjugates with two different chromogenic substrates for the enzymes in the ELISA assay, wherein the antigen is immobilized on a solid support during the sequential enzymatic reactions in an indirect, direct or competitive assay and wherein the at least two different analytes are each detected without interference in the presence of the analytes, enzyme labeled conjugates and chromogenic substrates.

The present invention also relates to a method for detecting at least two different antigens in a single enzyme-linked immunosorbent assay (ELISA) which comprises:

(a) providing a solid support which is capable of directly binding the analytes;

(b) providing enzyme labeled antibodies which are capable of binding to each of the antigens bound to the solid support;

(c) contacting the antigens bound to the solid support with the enzyme labeled antibodies; and

(d) detecting whether the sample contains each of the analytes by sequentially adding a chromogenic substrate specific for each of the enzyme labeled antibodies to be detected to produce chromogens which are detected, wherein the at least two different analytes are detected without interference in the presence of different of the analytes, enzyme labeled antibodies and the chromogenic substrates.

The present invention also relates to a method for detecting at least two analyte species in a single enzyme linked immunosorbent assay which comprises:

(a) providing a solid support which is capable of directly binding analyte;

(b) providing a first antibody which selectively binds to each of the analytes;

(c) providing anti-first antibody second antibodies each labeled with a different enzyme;

(d) contacting each of the analytes bound to the support with the first antibodies to produce first complexes;

(e) contacting the first complexes with the second antibodies each labeled with the different enzymes to produce second complexes; and

(f) detecting whether the sample contains each of the second complexes by sequentially adding different chromogenic substrates specific for each of the enzyme labels of each of the second antibodies to be detected to produce chromogens, wherein the at least two different analytes are each detected without interference in the presence of the analytes, enzyme labeled conjugates (antibodies) and chromogenic substrates.

The present invention further relates to a method for detecting two or more analyte species in a single enzyme-linked immunosorbent assay (ELISA), which comprises:

(a) providing for each analyte species to be detected, an antibody specific for the analyte species immobilized on a solid support;

(b) contacting the antibodies immobilized on the solid support to a liquid sample suspected of containing at least one of the analyte species for a time sufficient for the antibodies to bind the analyte species;

(c) removing the solid support from the liquid sample and washing the solid support to remove unbound material;

(d) contacting the solid support to a solution comprising for each analyte species to be detected, an antibody specific for the analyte species to be detected conjugated to an enzyme label wherein the enzyme label for each antibody is different for a time sufficient for the antibodies to bind the analyte species bound by the immobilized antibodies;

(e) removing the solid support from the solution and washing the solid support to remove unbound antibodies; and

(f) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

The present invention further relates to a method for detecting two analytes in a single enzyme-linked immunosorbent assay (ELISA), which comprises:

(a) providing a first antibody specific for a first analyte and a second antibody specific for a second analyte immobilized on a solid support;

(b) contacting the antibodies immobilized on the solid support to a liquid sample suspected of containing one or both of the analytes for a time sufficient for the antibodies to bind the analytes;

(c) removing the solid support from the liquid sample and washing the solid support to remove unbound material;

(d) contacting the solid support to a solution comprising a third antibody specific for the first analyte and a fourth antibody specific for the second analyte wherein the third antibody is conjugated to a first enzyme label and the fourth antibody is conjugated to a second enzyme label for a time sufficient for the third and fourth antibodies to bind the analytes bound by the first and second antibodies;

(e) removing the solid support from the solution and washing the solid support to remove unbound antibodies;

(f) adding a first chromogenic substrate for the first enzyme label wherein conversion of the first chromogenic substrate to a detectable color by the first enzyme label indicates that the sample contains the first analyte;

(g) removing the first chromogenic substrate; and

(h) adding a second chromogenic substrate for the second enzyme label wherein conversion of the second chromogenic substrate to a detectable color by the second enzyme label indicates that the sample contains the second analyte.

The present invention further relates to a method for determining whether a plant material is derived from a transgenic plant which comprises one or more heterologous genes by detecting the products produced by the one or more heterologous genes, which comprises:

(a) providing a liquid sample from the plant material;

(b) providing a solid support having a mixture of antibodies immobilized thereon wherein the mixture comprises antibodies specific for the products produced by the one or more heterologous genes;

(c) contacting the antibodies immobilized on the solid support to the liquid sample for a time sufficient for the antibodies to bind the products;

(d) removing the solid support from the liquid sample and washing the solid support to remove unbound material;

(e) contacting the solid support to a solution comprising for each product to be detected, an antibody specific for the product to be detected conjugated to an enzyme label wherein the enzyme label for each antibody is different for a time sufficient for the antibodies to bind the products bound by the immobilized antibodies;

(f) removing the solid support from the solution and washing the solid support to remove unbound antibodies; and

(g) determining whether the sample contains each product by sequentially detecting the enzyme labels by adding a chromogenic substrate specific for the enzyme label to be detected wherein conversion of the chromogenic substrate to a detectable color indicates the sample contains the product.

The present invention further relates to a kit for an ELISA comprising:

(a) a microtiter plate having a multiplicity of wells, each well having immobilized therein a mixture of two or more antibody species wherein each antibody species is specific for a particular analyte species;

(b) two or more first containers, each first container containing an antibody species conjugated to a particular enzyme label, wherein each antibody species is specific for the particular analyte species; and

(c) two or more second containers, each second container containing a chromogenic substrate, wherein each chromogenic substrate is specific for the particular enzyme label.

The present invention further relates to a method for detecting two or more analyte species in a single enzyme-linked immunosorbent assay (ELISA), which comprises:

(a) providing for each analyte species to be detected, an antibody specific for the analyte species immobilized on a solid support;

(b) providing a solution comprising for each analyte species to be detected, an antibody specific for the analyte species to be detected conjugated to an enzyme label wherein the enzyme label for each antibody is different;

(c) contacting a mixture comprising a sample suspected of containing at least one of the analyte species and the solution with the antibodies immobilized on the solid support for a time sufficient for the antibodies to bind the analyte species;

(d) removing the solid support from the mixture and washing the solid support to remove unbound antibodies; and

(e) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

The present invention further relates to a method for determining whether a plant material is derived from a transgenic plant which comprises one or more heterologous genes by detecting the products produced by the one or more heterologous genes, which comprises:

(a) providing a liquid sample from the plant material;

(b) providing a solid support having a mixture of antibodies immobilized thereon wherein the mixture comprises antibodies specific for the products produced by the one or more heterologous genes;

(c) providing a solution comprising for each product to be detected, an antibody specific for the product to be detected conjugated to an enzyme label wherein the enzyme label for each antibody is different for a time sufficient for the antibodies to bind the products bound by the immobilized antibodies to produce a mixture;

(d) contacting the antibodies immobilized on the solid support to a mixture comprising the sample and the solution for a time sufficient for the antibodies to bind the products;

(e) removing the solid support from the mixture and washing the solid support to remove unbound material; and

(f) determining whether the sample contains each product by sequentially detecting the enzyme labels by adding a chromogenic substrate specific for the enzyme label to be detected wherein conversion of the chromogenic substrate to a detectable color indicates the sample contains the product.

The present invention relates to a kit for an ELISA for two or more analytes in a single assay comprising:

(a) a solid support having immobilized therein a mixture of two different antibodies specific for the analytes or purified analytes, wherein each of the antibodies is specific for a particular one of the analytes;

(b) one or more first containers each of the first containers containing second antibodies each labeled with different enzymes, which second antibodies are specific for each of the analytes; and

(c) two or more second containers, each of the two second containers containing a chromogenic substrate which is specific for each of the enzyme labels, wherein the analytes and antibodies are non-interfering in the assay.

The present invention further relates to a kit for an ELISA for two or more analytes in a single assay comprising:

(a) a solid support having immobilized thereon a mixture of two different first antibodies or a purified analyte, wherein each of the antibodies is specific for a particular one of the analytes;

(b) one or more first containers containing second antibodies which antibodies are specific for each of the analytes;

(c) one or more second containers, each of the second containers containing anti-antibody second antibodies each conjugated to a different enzyme label which binds to the second antibodies; and

(d) two or more third containers containing a chromogenic substrate which is specific for each of the enzyme labels, wherein the analytes and antibodies are non-interfering in the assay.

The present invention provides a multiplex enzyme-linked immunosorbent assay (ELISA) for detecting multiple analytes. In the present invention, a single ELISA is used to detect one or more analytes or antibodies in a single assay by using enzyme-labeled analytes or antibodies wherein the analyte or antibodies for detecting each analyte or antibody is labeled with a different enzyme. Therefore, by using a combination of antibodies conjugated to different enzymes, a simple multi-analyte ELISA can be done in a single well of a plate. The technique utilizes the availability of different chromogenic substances that are specific to the respective enzymes. Enzymes commonly used in ELISAs include alkaline phosphatase, horseradish peroxidase (peroxidase), β-galactosidase, β-glucoronidase, urease, and penicillinase. The present invention includes direct ELISAs for detecting an antigen in a sample, indirect ELISAs for detecting an antibody in a sample, direct competitive ELISAs for detecting an analyte in a sample, and indirect competitive ELISAs for detecting an antibody in a sample. The present invention further includes kits comprising ELISAs for detecting multiple analytes or antibodies in a sample.

Several of the advantages of the present invention include (1) a single plate can be used to detect more than one analyte, (2) less sample volume is used in the assay, (3) less reagent volume is used in the assay, (3) the assay can be adapted to automated protocols, (4) a single plate can be used to detect diseases which have more than one determinant or marker, (5) a single plate can be used to detect recombinant organisms, including transgenic organisms, which express more than one determinant or marker, and (6) a single plate can be used to detect more than one pathogen in a sample; and (7) there are fewer steps and thus fewer possible technician errors.

In one embodiment, the present invention provides a direct ELISA for detecting multiple antigens in a sample wherein for each analyte to be detected by the ELISA, antibodies specific for the analyte are immobilized as a mixture on a solid support or surface by methods well known in the art. Preferably, the antibodies for each analyte to be detected are immobilized as a mixture in the wells of a microtiter plate which is commonly used for ELISA assays. Next, a sample is added to the wells containing the immobilized antibodies and allowed to incubate in the wells for a time sufficient for each of the analytes in the sample to bind to the appropriate immobilized antibody. The sample can be provided neat or in a limiting dilution series in a physiological solution, or any other suitable buffer. Unbound material in the sample is removed from the immobilized antibody-analyte complexes by washing. The complexes are then reacted with a mixture of second antibodies, each antibody in the mixture specific for one of the analytes or analyte-antibody complexes and conjugated to (labeled with) a particular enzyme label such that detection of an analyte-antibody complex is determined by measuring the activity of the particular enzyme conjugated to the antibody for binding to the analyte or analyte-antibody complex. After incubating for a time sufficient to allow the antibodies in the mixture to bind the analyte or analyte-antibody complex, the mixture is removed by washing. Detection of the enzyme label is by adding to the well a substrate for the enzyme. The amount of each analyte species in the sample is directly proportional to the signal strength. In practice, detection of the labels are done in separate reactions.

The above embodiment where the sample is added to the immobilized antibody and then removed before adding the antibody conjugates is called a “sequential assay”. In particular aspects of the above embodiment, a “cocktail” assay is performed. In this aspect, the antibody conjugates are mixed with the sample and the mixture is then contacted to the immobilized antibody. After removing the mixture and washing, detection of each conjugate species is performed sequentially. In some further embodiments of the equilibrium assay, the sample and antibody conjugates are separately added to the immobilized antibody and the resulting mixture incubated as above.

The above embodiment where the sample is mixed with the immobilized analyte and then removed before mixing with the antibody conjugates is called a competition assay. In particular aspects of the above “competition” embodiment, an equilibrium assay is performed. In this aspect, competition antibodies are mixed with the sample and the mixture is then contacted with the immobilized analyte. After removing the mixture and washing, detection of each conjugate species is performed sequentially. In some further embodiments of the equilibrium assay, the sample and competition antibodies are separately added to the immobilized analyte and the resulting mixture incubated as above.

In another embodiment, the present invention provides a competitive ELISA for detecting multiple analytes in a sample wherein for each analyte to be detected by the ELISA, antibodies specific for the analyte are immobilized as a mixture on a solid support or surface by methods well known in the art. Preferably, the antibodies for each analyte to be detected are immobilized as a mixture in the wells of a microtiter plate which is commonly used for ELISA assays. Next, enzyme-labeled analyte (separate species of enzyme for each analyte species) is mixed with a sample and the mixture is then added to the wells containing the immobilized antibodies for a time sufficient for the analytes in the sample to bind to the appropriate immobilized antibody. The sample can be provided neat or in a limiting dilution series in a suitable buffer. Unbound material in the sample is removed from the immobilized antibody-analyte complexes by washing. Detection of the enzyme label is by adding to the well a substrate for the enzyme. The amount of each analyte species in the sample is inversely proportional to the signal strength. In practice, detection of the labels are done in separate reactions.

In another embodiment, the present invention provides a competitive indirect ELISA for detecting multiple analytes in a sample wherein each analyte to be detected by the ELISA, is immobilized as a mixture on a solid support or surface by methods well known in the art. Preferably, the analytes are immobilized as a mixture in the wells of a microtiter plate which is commonly used for ELISA assays. Next, enzyme-labeled antibody (separate species of enzyme for each antibody species) is mixed with a sample and the mixture is then added to the wells containing the immobilized analyte for a time sufficient for the antibodies in the sample to bind to the appropriate immobilized analyte. The sample can be provided neat or in a limiting dilution series in a suitable buffer. Unbound material in the sample is removed from the immobilized antibody-analyte complexes by washing. Detection of the enzyme label is by adding to the well a substrate for the enzyme. The amount of each antibody species in the sample is inversely proportional to the signal strength. In practice, detection of the labels is done in separate reactions.

Preferred enzyme labels include alkaline phosphatase, horseradish peroxidase (peroxidase), β-galactosidase, β-glucoronidase, urease, and penicillinase. Suitable chromogenic substrates for peroxidase-linked assays include, but are not limited to, o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), 3,3′,5,5′-tetramethylbenzidine (TMB), and other soluble chromogenic substrates suitable for peroxidase-linked ELISAs.

Suitable chromogenic substrates for alkaline phosphatase-linked assays include bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT) (BPA and B available as BLUEPHOS from KPL, Inc., Gaithersburg, Md. and disclosed in U.S. Pat. No. 5,916,746 to Cobb et al.), pNPP or PNP (para-nitrophenyl phosphate) and other soluble chromogenic substrates suitable for alkaline phosphatase-linked ELISAs.

Suitable chromogenic substrates for penicillinase-linked assays include starch-iodine-penicillin V, bromothymol blue-penicillin V, bromothymol blue (BTB), bromochloroindolyl phosphate (BCP), and other soluble chromogenic substrates suitable for penicillinase-linked ELISAs.

Suitable chromogenic substrates for β-galactosidase include ONPG and other soluble chromogenic substrates suitable for β-galactosidase-linked ELISAs. Suitable chromogenic substrates for β-glucoronidase include phenolphthalein and other soluble chromogenic substrates suitable for β-glucoronidase-linked ELISAs.

Urease catalyzes hydrolysis of urea into ammonia and carbon dioxide. Urease substrates are commercially available which change color from yellow to purple, which can be read at 590 nm.

In further embodiments, one or more of the antibodies in the mixture can be conjugated to a reporter ligand such as a fluorescing ligand, chemiluminescent ligand, biotin, colored latex, colloidal gold magnetic beads, radioisotopes, or the like. Detection of the complex is by methods well known in the art for detecting the particular reporter ligand.

The following is a list of important bacterial, viral, and fungal plant pathogens for which various embodiments of the multiplex ELISA of the present invention are useful.

### Plant Viruses

African Cassaya Mosaic Virus (CMV)

Alfalfa Mosaic Virus (AMV)

Alstroemeria Mosaic Virus (AlMV)

American Plum Line Pattern Virus (APLPV)

Andean Potato Latent Virus (APLV)

Apple Chlorotic Leaf Spot Virus (ACLSV)

Apple Mosaic Virus (ApMV)

Arabis Mosaic Virus (ArMV)

Asparagus Virus 2 (AV2)

Banana Bract Mosaic Virus (BbrMV)

Banana Bunchy Top Virus (BBTV)

Banana Streak Virus (BSV)

Barley Stripe Mosaic Virus (BSMV)

Barley Yellow Dwarf Virus-may (BYDV-may)

Barley Yellow Dwarf Virus-pav (BYDV-pav)

Barley Yellow Dwarf Virus-rmv (BYDV-rmv)

Barley Yellow Dwarf Virus-rpv (BYDV-rpv)

Barley Yellow Dwarf Virus-sgv (BYDV-sgv)

Bean Common Mosaic Virus (BCMV)

Bean Pod Mottle Virus (BPMV)

Bean Yellow Mosaic Virus (BYMV)

Beet Necrotic Yellow Vein Virus BNYVV Beet Western

Yellows Virus (BWYV)

Blueberry Leaf Mottle Virus (BLMV)

Blueberry Shock Virus (BlShV)

Blueberry Scorch Virus (BBScV)

Blueberry Shoestring Virus (BSSV)

Broad Bean Wilt Virus (BBWV)

Brome Mosaic Virus (BMV)

Calibrachoa Mottle Virus (CbMV)

Carnation Etched Ring Virus (CERV)

Carnation Latent Virus (CLV)

Carnation Mottle Virus (CarMV)

Carnation Necrotic Fleck Virus (CNFV)

Carnation Ringspot Virus (CRSV)

Cauliflower Mosaic Virus (CaMV)

Cherry Leaf Roll Virus (CLRV)

Chrysanthemum Chlorotic Mottle Viroid (CChMVd)

Chrysanthemum Stunt Viroid (CSVd)

Chrysanthemum Virus B (CVB)

Citrus Tristeza Virus (CTV)

Cowpea Mosaic Virus (CPMV)

Cucumber Green Mottle Mosaic Virus (CGMMV)

Cucumber Mosaic Virus (CMV)

Cucumber Mosaic Virus-Subgroup I (CMV I)

Cucumber Mosaic Virus-Subgroup II (CMV II) Cymbidium

Mosaic Virus (CyMV)

Cymbidium Ringspot Virus (CyRSV)

Dasheen Mosaic Virus (DsMV)

Garlic Common Latent Virus (GCLV)

Geminiviruses-3F7 (3F7)

Grapevine Fanleaf Virus (GFLV)

Grapevine Fleck Virus (GFkV)

Grapevine Leaf Roll Virus 1 (GLRV 1)

Grapevine Leaf Roll Virus 2 (GLRV 2)

Grapevine Leaf Roll Virus 3 (GLRV 3)

Grapevine Leaf Roll Virus 5 (GLRV 5)

Grapevine Leaf Roll Virus 7 (GLRV 7)

Grapevine Virus A (GVA)

Groundnut Bud Necrosis Virus (GBNV)

Groundnut Ringspot Virus (GRSV)

Hibiscus Chlorotic Ringspot Virus (HCRSV)

Hosta Virus X (HsVX)

Impatiens Necrotic Spot Virus (INSV)

Iris Yellow Spot Virus (IYSV)

Johnsongrass Mosaic Virus (JgMV)

Kalanchoe Latent Virus (KLV)

Kyuri Green Mottle Mosaic Virus (KGMMV)

Leek Yellow Stripe Virus (LYSV)

Lettuce Mosaic Virus (LMV)

Lily Symptomless Virus (LSV)

Maize Chlorotic Mottle Virus (MCMV)

Maize Dwarf Mosaic Virus (MDMV)

Maize Mosaic Virus (MMV)

Maize Stripe Virus (MSPV)

Maize White Line Mosaic Virus (MWLMV)

Melon Necrotic Spot Virus (MNSV)

Nandina Mosaic Virus (NaMV)

Odontoglossum Ringspot Virus (ORSV)

Onion Yellow Dwarf Virus (OYDV)

Papaya Mosaic Virus (PapMV)

Papaya Ringspot Virus (PRSV)

Pea Seed-borne Mosaic Virus (PSbMV)

Peach Rosette Mosaic Virus (PRMV)

Peanut Stunt Virus (PSV)

Pelargonium Flower Break Virus (PFBV)

Pelargonium Leaf Curl Virus (PLCV)

Pelargonium Zonate Spot Virus (PZSV)

Pepino Mosaic Virus (PepMV)

Pepper Mild Mottle Virus (PMMOV)

Pepper Mottle Virus (PepMoV)

Plum Pox Virus (PPV)

Poinsettia Mosaic Virus (PnMV)

Potato Aucuba Mosaic Virus (PAMV)

Potato Latent Virus (PotLV)

Potato Leaf Roll Virus (PLRV)

Potato Spindle Tuber Viroid (PSTVd)

Potato Virus A (PVA)

Potato Virus M (PVM)

Potato Virus S (PVS)

Potato Virus T (PVT)

Potato Virus V (PVV)

Potato Virus X (PVX)

Potato Virus Y (PVY)

Potato Virus Y-necrotic strain (PVY-n)

Potato Virus Y-strain-o (PVY-o)

Potyvirus Group (POTY)

Prune Dwarf Virus (PDV)

Prunus Necrotic Ringspot Virus (PNRSV)

Raspberry Bushy Dwarf Virus (RBDV)

Raspberry Ringspot Virus (RPRSV)

Red LaSoda Virus (RLaSV)

Ribgrass Mosaic Virus (RMV)

Shallot Latent Virus (SLV)

Shallot Yellow Stripe Virus (SYSV)

Soil-borne Wheat Mosaic Virus (SBWMV)

Southern Bean Mosaic Virus (SBMV)

Soybean Mosaic Virus (SMV)

Squash Mosaic Virus (SqMV)

Stolbur MLO (SMLO)

Strawberry Latent Ringspot Virus (SLRSV)

Strawberry Mild Yellow Edge-associated Virus (SMYEaV)

Sugarcane Bacilliform Virus (SCBV)

Sugarcane Mosaic Virus (SCMV)

Tobacco Etch Virus (TEV)

Tobacco Mosaic Virus (TMV)

Tobacco Mosaic Virus-c (TMV-c)

Tobacco Rattle Virus (TRV)

Tobacco Ringspot Virus (TRSV)

Tobacco Streak Virus (TSV)

Tobacco Vein Mottling Virus (TVMV)

Tomato Aspermy Virus (TAV)

Tomato Black Ring Virus-S, G (TBRV)

Tomato Bushy Stunt Virus (TBSV)

Tomato Chlorotic Spot Virus (TCSV)

Tomato Mosaic Virus (TOMV)

Tomato Ringspot Virus (TORSV)

Tomato Spotted Wilt Virus (TSWV)

Tospovirus Group (TOSPO)

Turnip Mosaic Virus (TuMV)

Watermelon Mosaic Virus 2 (WMV2)

Wheat Dwarf Virus (WDV)

Wheat Spindle Streak Mosaic Virus (WSSMV)

Wheat Streak Mosaic Virus (WSMV)

Zucchini Yellow Mosaic Virus (ZYMV)

### Plant Bacteria

Acidovorax avenae subsp. citrulli (Aac)

Clavibacter michiganensis subsp. michiganensis (Cmm)

Clavibacter michiganensis subsp. nebraskensis (Cmn)

Clavibacter michiganensis subsp. sepedonicus (Cms)

Clavibacter michiganensis subsp. tessellarius (Cmt)

Corn Stunt Spiroplasma (CSS)

Erwinia amylovora (Ea)

Erwinia carotovora (Ec)

Erwinia carotovora subsp. atroseptica (Eca)

Erwinia chrysanthemi (Echr)

Erwinia stewartii (Es)

Pseudomonas avenae (Pa)

Pseudomonas fuscovaginae (Pf)

Pseudomonas glumae (Pg)

Pseudomonas syringae pv. phaseolicola (Psph)

Ralstonia solanacearum (Rs)

Spiroplasma citri (Sc)

Xanthomonas (Xan)

Xanthomonas albilineans (Xalb)

Xanthomonas campestris pv. armoraciae (Xcarm)

Xanthomonas campestris pv. begoniae (Xcb)

Xanthomonas campestris pv. campestris (Xcc)

Xanthomonas axonopodis pv. citri (Xccit or Xacit)

Xanthomonas campestris pv. dieffenbachiae (Xcd)

Xanthomonas campestris pv. oryzae (Xco)

Xanthomonas campestris pv. pelargonii (Xcp)

Xanthomonas campestris pv. phaseoli (Xcph)

Xanthomonas campestris pv. vesicatoria (Xcv)

Xanthomonas maltophilia (Xm)

Xylella fastidiosa (Xf)

### Plant Fungi

Phytophthora (Phyt)

Verticillium dahlia (Vdah)

Other plant viruses not mentioned herein are recited in the VIDE database published by CAB International Publishing, New York, N.Y. Other plant bacteria not mentioned herein are catalogued at the national Collection of Plant Bacteria at the Central Science Laboratory, Sand Hutton, York, United Kingdom. Other plant fungi not mentioned herein are recited in the U.S. National Fungus Collections database, Agricultural Research Service, Beltsville, Md.

The present invention is also useful for providing multiplex ELISAS, for identifying transgenic organisms such as transgenic plants by the particular products produced by the transgenic organism. Examples of such products in the case of transgenic plants include the Bt-Cry1Ab, the Bt-Cry1Ac, Bt-Cry3Bb1, BtCry1F, BtCry2A, Bt-Cry3A, Bt-Cry9C, phosphinothricin acetyltransferase (PAT), 5-enolpyruvylshikimate-3-phosphate synthase (EPSPS), and neomycin phosphotransferase II (NPTII), a common marker used for making transgenic plants.

The present invention is useful for providing multiplex ELISAs for identifying various pests, in particular, plant pests such as the cotton bollworm and the tobacco budworm.

The present invention is useful for providing multiplex ELISAs for detecting and quantifying hormones produced by an organism, in particular, hormones produced by a plant such as plant growth hormones such as abscisic acid, dihydrozeatin riboside, indole-3-acetic acid, isopentenyladenosine, or trans-zeatin riboside.

There are a variety of transgenic plants which contain more than one transgene. Many times it is important to determine whether particular seeds, seedlings, or plant material is from or of the transgenic plant. An incomplete list of examples of transgenic plants which contain more than one heterologous gene include the following.

Insect-resistant and bromoxynil herbicide tolerant cotton produced by inserting the cry1Ac gene from Bacillus thuringiensis and a nitrilase encoding gene from Klebsiella pneumoniae.

Insect-resistant and glyphosate herbicide tolerant cotton produced by inserting the cry1Ac gene from Bacillus thuringiensis and a gene encoding a naturally glyphosate tolerant form of the enzyme 5-enolpyruvyl shikimate-3-phosphate synthase (EPSPS) from A. tumefaciens strain CP4.

Insect-resistant and glufosinate ammonium herbicide tolerant cotton produced by inserting the cry1Ac gene from Bacillus thuringiensis and inserting a modified phosphinothricin acetyltransferase (PAT) encoding gene from the soil bacterium Streptomyces hygroscopicus.

Insect-resistant cotton derived by transformation of the DP50B parent variety, which contains the gene encoding Cry1Ac from Bacillus thuringiensis, with purified plasmid DNA containing the cry2Ab gene from B. thuringiensis subsp. kurstaki.

Insect-resistant and glufosinate ammonium herbicide tolerant maize derived by inserting the crylF gene from Bacillus thuringiensis var. aizawai and the phosphinothricin N-acetyltransferase (PAT) encoding gene from Streptomyces viridochromogenes.

Insect-resistant and glufosinate ammonium herbicide tolerant maize derived by inserting genes encoding Cry3Bb1 protein from Bacillus thuringiensis subsp kurstaki and phosphinothricin acetyltransferase (PAT) from Streptomyces hygroscopicus.

Insect-resistant and glufosinate ammonium herbicide tolerant maize derived by inserting genes encoding Cry1AC protein from Bacillus thuringiensis subsp kurstaki and phosphinothricin acetyltransferase (PAT) from Streptomyces hygroscopicus.

Insect-resistant and glufosinate ammonium herbicide tolerant maize derived by inserting genes encoding Cry9C protein from Bacillus thuringiensis subsp tolworthi and phosphinothricin acetyltransferase (PAT) from Streptomyces hygroscopicus.

Insect-resistant and herbicide tolerant maize derived by inserting the cry1Ab gene from Bacillus thuringiensis subsp. kurstaki, and the phosphinothricin N-acetyltransferase (PAT) encoding gene from S. viridochromogenes.

Colorado potato beetle and potato leafroll virus (PLRV) resistant potatoes derived by inserting the cry3A gene from Bacillus thuringiensis (subsp. Tenebrionis) and the replicase encoding gene from PLRV.

Colorado potato beetle and potato virus Y (PVY) resistant potatoes derived by inserting the cry3A gene from Bacillus thuringiensis (subsp. Tenebrionis) and the coat protein encoding gene from PVY.

Cucumber mosaic virus (CMV), zucchini yellows mosaic (ZYMV) and watermelon mosaic virus-2(WMV-2) resistant squash (Curcurbita pepo) derived by inserting the coat protein encoding sequences from each of these plant viruses into the host genome.

Zucchini yellows mosaic (ZYMV) and watermelon mosaic virus (WMV) 2 resistant squash (Curcurbita pepo) derived by inserting the coat protein encoding sequences from each of these plant potyviruses into the host genome.

Preparing samples for the multiplex ELISA disclosed herein can use the methods which follow. Other methods for preparing plant samples for ELISAs are well known in the art and can be used in lieu of the methods recited below.

Leaves, seedlings, or seeds from transgenic plants or infected plants are ground and diluted in MEB sample extraction buffer or other suitable buffers at a defined ratio of sample to buffer listed in Table 1. MEB buffer is 1×PBST containing 0.4% non-fat dried milk and 0.5% TWEEN 20. 1×PBST contains 8.2 mM dibasic sodium phosphate, 2.7 mM potassium chloride, 1.5 mM monobasic potassium phosphate, and 137 mM sodium chloride. After samples have been ground in buffer, let the extract sit for at least 30 seconds.

TABLE 1

| | LEAF to MEB buffer ratio | SEED to MEB buffer ratio |
|---|---|---|
| Crop | (weight/volume) | (weight/volume) |
| Corn | 1:10 | 1:10 |
| Cotton | 1:20 | 1:20 |
| Soybean | 1:20 | 1:20 |

Leaf Extraction:

For leaf samples use disposable sample extraction bags available from Agdia, Inc., Elkhart, Indiana, a clean mortar and pestle, or any other grinding device to help extract samples.

Individual Leaves:

A simple method for grinding a single leaf sample is by using Agdia's sample extraction bags. Use only one sample per bag and be sure to label each bag. Add the appropriate volume of buffer to an empty bag. A recommended 1:20 dilution, would require a 0.15 g leaf sample and 3 mL of buffer. Place the sample between the mesh linings of the pouch. Rub the pouch with a pen to completely crush the sample and to mix the contents uniformly.

Multiple Leaves:

For composite leaf samples (up to 100 leaves), taking a representative leaf disc or leaf punch is recommended. Stack the leaves on a clean surface and using a No. 2 cork borer, punch through the leaves to produce 100 leaf discs. Dislodge the discs from the cork borer with a clean metal wire, weigh and transfer the discs into the sample extraction bags and extract in buffer according to the recommended ratios in Table 2. The weight of the discs varies with the growing conditions, age, and variety of the plant. Determine the average weight of the leaf discs and add the appropriate volume of buffer.

TABLE 2

| | LEAF to MEB | | |
|---|---|---|---|
| | buffer ratio | Approximate weight | Volume of MEB |
| Crop | (weight/volume) | of 100 discs | Buffer |
| Corn | 1:10 | 0.2 g | 2 mL |
| Cotton | 1:20 | 0.2 g | 4 mL |
| Soybean | 1:20 | 0.1 g | 2 mL |

Seed Extraction:

Single Seeds:

Single seeds can be crushed with a seed crusher or hammer. Determine the average weight of the seed and add the appropriate volume of MEB buffer. Let the extract sit for at least 30 seconds before testing with the ELISA.

Multiple Seeds:

For seed samples to be tested at 0.1% sensitivity level, it is recommended to use a blender with “Mason” type jars to accommodate 1000 seeds. However, depending on the sample size other devices like coffee grinders, ball mill, other blenders, or seed crusher may be used to grind the samples. The guidelines provided are optimized for blenders with “Mason” type jars.

Put the seed sample in a dry “Mason” jar and assemble the blade attachment. Grind the seed at high speed for about 45-60 seconds or until all the seeds are ground to a powder. Remove the jar from the blender and tap to collect all the powder. Shake the jar to mix and check for any unground seed. Transfer the ground powder to a container and weigh the specified amount (sub sample) from Table 3 to a 500 mL disposable bottle. Add the buffer at the specified ratio, close the lid and shake the bottle for 10-15 seconds. Let the extract sit for at least 30 seconds before testing with the ELISA. Use only the supernatant (top layer of liquid) for testing.

TABLE 3

| | Seed to MEB buffer ratio | Sub sample | Volume of MEB |
|---|---|---|---|
| Crop | (weight/volume) | weight | Buffer |
| Corn | 1:10 | 50 g | 500 mL |
| Cotton | 1:20 | 20 g | 400 mL |
| Soybean | 1:20 | 20 g | 400 mL |

Positive and Negative Controls:

Reconstitute the bottle of lyophilized positive control and negative control with 2.5 ml MEB sample extract buffer. The concentration of the reconstituted control is about 1% CP4 EPSPS seed.

After preparing the positive and negative controls, divide them into aliquots, each sufficient for one use. Dispense aliquots into tubes that can be securely capped. Dispense 120 μL if one well is used for positive control or 220 μL if two wells are used per test. Each aliquot should be sufficient for the tests to be run plus a small additional volume to assure easy dispensing.

Control aliquots must be stored frozen (−20° C. freezer or household freezer). Do not thaw until just before use. At the time of each test run, remove from storage only the aliquots that will be used. Allow the tubes to thaw, then mix the contents thoroughly. At the time you add sample extracts to test wells, add the same volume of negative and positive control to the appropriate control wells.

The following is a general scheme for using a multiplex-ELISA for two target antigens, A and B. The first step involves separate optimization of individual ELISA tests for A and B targets. The primary requisite is that the detection antibodies (enzyme conjugates) should be specific to their respective antigens and not cross react with heterologous antigens.

The primary antibodies (capturing antibodies) are then mixed at a specific optimized ratio and are then coated (immobilized) to the wells of a polystyrene ELISA plate in a buffer such as carbonate-bicarbonate (pH 9.5) buffer. The reactive binding sites on the plate are then blocked with a protein buffer (1% BSA in PBS pH 7.4 or 1% BSA in carbonate-bicarbonate buffer, for example). Samples which might contain the multiple targets A and B are then added to the ELISA plate wells and incubated at room temperature for a time sufficient for the immobilized antibodies to bind their respective targets (usually an hour is sufficient). In general, about 100 μL of sample is added per well. A useful buffer to use is MEB buffer; however, other buffers can also be used. Afterwards, the samples are removed and the wells washed with a buffer such as 1×PBST. Preferably, the wells are washed 3 to 7 times.

Next, a mixture of labeled anti-A and anti-B antibodies conjugated to two different reporter enzymes, for example, alkaline phosphatase is conjugated to anti-A and a peroxidase is conjugated to anti-B, in a common buffer that is compatible for the two enzymes is added to each of the wells (about 100 μL/well). An example of a compatible buffer is ECM buffer (1×PBST containing 0.4% non-fat dried milk) (Agdia, Elkhart, Ind.). After incubating at room temperature for a time sufficient for the labeled antibodies to bind their respective targets (usually an hour is sufficient), the wells are washed as above to remove the unbound enzyme conjugates. In some instances the antibodies are not in a mixture but are applied sequentially with an intervening wash.

Next, a chromogenic substrate for the alkaline phosphatase is added to the wells. For example, pNPP substrate (para-nitrophenyl phosphate) is added to the wells following the instructions of the manufacturer (about 100 μL/well). The anti-A alkaline phosphatase conjugate generates a signal by changing the color of the pNPP substrate to yellow which can be recorded at 405 nm after 30 to 60 minutes. The wells are then washed as above and TMB (tetramethylbenzidine) substrate added to the wells. The peroxidase oxidizes the TMB to a blue color which can be recorded at 650 nm after 20 to 30 minutes. In some instances, an acid stop such as 3M sulfuric acid can be added and the optical density of the resulting yellow color read at 450 nm.

While in particular embodiments the TMB can added prior to the pNPP, in preferred embodiments, the TMB always follows the alkaline phosphatase substrate. Further embodiments include adding the sample and enzyme conjugate one after another (sequential ELISAs) or together (cocktail ELISAs). A further still embodiment includes a different substrate for alkaline phosphatase, such as the soluble BCIP substrate (available as BLUEPHOS from KPL, Inc., Gaithersburg, Md.) or pNPP followed by a different substrate for the peroxidase, such as OPD (o-phenylenediamine).

In further embodiments, the ELISA can be used to detect a third target C by using antibodies to target C which are labeled with an enzyme such as β-galactosidase and detecting the β-galactosidase with the substrate ONPG (o-nitrophenyl-β,D-galactopyranoside). The ONPG can be detected at 405 nm under alkaline conditions after the TMB-peroxidase reaction has been performed. In further still embodiments, a fourth target D is detected using antibodies against target D are labeled with the enzyme penicillinase. The penicillinase is detected using a chromogenic substrate comprising penicillin and a suitable pH indicator.

The following examples are intended to promote a further understanding of the present invention.

### EXAMPLE 1

This example illustrates a multiplex ELISA for detecting BtCry3Bb1 (3Bb1) and BtCry1Ab/1Ac (1Ab/1Ac) in a sample.

A mixture of polyclonal antibodies against 3Bb1 and 1Ab/1Ac at a ratio of 2 to 1 and a mixture at a ratio of 2 to 1.5 were each coated to the bottom of the wells of an ELISA plate, plates A and B respectively, using standard methods for coating ELISA plate wells with antibodies. The antibodies are available from Agdia, Inc., Elkhart, Ind.

A sample containing 3Bb1 protein (C1133) in MEB buffer and a sample containing 1Ab/1Ac protein (C1087) in MEB buffer were each serially diluted 1:1, 1:2, 1:4, 1:8, 1:16, and 1:32. BtCry3Bb1 and 1Ab/1Ac are available from Agdia, Inc. MEB buffer is 1×PBST containing 0.4% non-fat dried milk and 0.5% TWEEN 20. 1×PBST contains 8.2 mM dibasic sodium phosphate, 2.7 mM potassium chloride, 1.5 mM monobasic potassium phosphate, and 137 mM sodium chloride.

Each dilution was added to two wells of each of the above ELISA plates prepared as above to produce first and second replicates of each dilution for each plate (100 μL/well). An MEB buffer control and a negative control was included for each replicate. The plates were then incubated at room temperature for about an hour. Afterwards, dilutions were removed from the wells and the wells washed with 1×PBST at room temperature about 6 to 7 times. The wells were soaked about 3 minutes in 1×PBST and then the 1×PBST was decanted.

Next, a mixture of antibody specific for 1Ab/1Ac and conjugated to a peroxidase and antibody specific for 3Bb1 and conjugated to alkaline phosphatase was added to each of the wells. The antibodies were at an appropriate ratio in MRS buffer (1×PBS TWEEN buffer containing 20% horse serum) and 100 μL was added per well. After about an hour at room temperature, the antibodies were removed and the wells washed with 1×PBST 6 to 7 times. The wells were soaked about 3 minutes in 1×PBST and then the 1×PBS was decanted. An alternative buffer for the antibodies is ECM which is 1×PBST containing 0.4% non-fat dried milk.

Next, to replicates 1 and 2, a solution of TMB (3,3′,5,5′-tetramethylbenzidine; Agdia, Inc.) was added (100 μL/well) according to the manufacturer's instructions and after 20-30 minutes at room temperature, the absorbance at 650 nm was read using an ELISA reader. To replicates 3 and 4, a solution of BPA and B (hereinafter “BP” which is provided as BLUEPHOS, a two-component chromogenic substrate available from KPL, Inc., Gaithersburg, Md.) was added according to the manufacturer's instructions (100 μL/well) and after 30-60 minutes at room temperature, the absorbance at 630 nm was read using an ELISA reader. Afterwards, all the wells were washed as above. Then, to replicates 1 and 2, a solution of BP was added and after 3-60 minutes at room temperature, the absorbance at 630 nm was read using an ELISA reader. To replicates 3 and 4, a solution of TMB was added and after 20-30 minutes at room temperature, the absorbance at 650 nm was read using an ELISA reader. The results are shown in Tables 4-7.

TABLE 4
Plate A: Absorbance read at 650 nm

| | | TMB Rxn First | | TMB Rxn Second | |
|---|---|---|---|---|---|
| | | BtCry3Bb | 1 Ab/1 Ac | BtCry3Bb1 | 1 Ab/1 Ac |
| Sample | Dilution | 1 | 2 | 3 | 4 |
| 3Bb1 or | 32 | 0.022 | 3.00 | 0.054 | 2.896 |
| 1Ab/1Ac | | | | | |
| | 16 | 0.020 | 2.746 | 0.039 | 2.784 |
| | 8 | 0.020 | 2.234 | 0.028 | 2.153 |
| | 4 | 0.022 | 1.530 | 0.018 | 1.367 |
| | 2 | 0.020 | 0.901 | 0.016 | 0.809 |
| | 1 | 0.021 | 0.496 | 0.015 | 0.446 |
| MEB | 0 | 0.023 | 0.014 | 0.018 | 0.016 |
| Neg. | 0 | 0.023 | 0.013 | 0.017 | 0.015 |
| control | | | | | |

TABLE 5
Plate A: Absorbance read at 630 nm

| | | AP Rxn Second | | AP Rxn First | |
|---|---|---|---|---|---|
| | | BtCry3Bb1 | 1 Ab/1 Ac | BtCry3Bb1 | 1 Ab/1 Ac |
| Sample | Dilution | 1 | 2 | 3 | 4 |
| 3Bb1 or | 32 | 1.423 | 0.035 | 2.391 | 0.045 |
| 1Ab/1Ac | | | | | |
| | 16 | 0.866 | 0.033 | 2.090 | 0.042 |
| | 8 | 0.377 | 0.032 | 1.687 | 0.043 |
| | 4 | 0.178 | 0.036 | 0.935 | 0.043 |
| | 2 | 0.100 | 0.035 | 0.483 | 0.043 |
| | 1 | 0.065 | 0.038 | 0.236 | 0.046 |
| MEB | 0 | 0.039 | 0.035 | 0.044 | 0.043 |
| Neg. | 0 | 0.043 | 0.034 | 0.045 | 0.044 |
| control | | | | | |

TABLE 6
Plate B: Absorbance read at 650 nm

| | | TMB Rxn First | | TMB Rxn Second | |
|---|---|---|---|---|---|
| | | BtCry3Bb1 | 1 Ab/1 | BtCry3Bb1 | 1 Ab/1 Ac |
| Sample | Dilution | 1 | Ac 2 | 3 | 4 |
| BtCry3Bb1 | 32 | 0.021 | 2.493 | 0.043 | 2.320 |
| or | | | | | |
| 1Ab/1Ac | | | | | |
| | 16 | 0.023 | 2.158 | 0.035 | 2.006 |
| | 8 | 0.020 | 1.670 | 0.026 | 1.457 |
| | 4 | 0.022 | 1.096 | 0.018 | 0.899 |
| | 2 | 0.021 | 0.659 | 0.015 | 0.521 |
| | 1 | 0.021 | 0.379 | 0.014 | 0.299 |
| MEB | 0 | 0.021 | 0.022 | 0.015 | 0.014 |
| Neg. | 0 | 0.021 | 0.023 | 0.014 | 0.014 |
| control | | | | | |

TABLE 7
Plate B: Absorbance read at 630 nm

| | | AP Rxn Second | | AP Rxn First | |
|---|---|---|---|---|---|
| | | BtCry3Bb | 1 Ab/1 Ac | BtCry3Bb | 1 Ab/1 Ac |
| Sample | Dilution | 1 | 2 | 3 | 4 |
| 3Bb1 or | 32 | 1.249 | 0.045 | 2.127 | 0.043 |
| 1Ab/1Ac | | | | | |
| | 16 | 0.650 | 0.041 | 1.849 | 0.042 |
| | 8 | 0.352 | 0.041 | 1.221 | 0.041 |
| | 4 | 0.155 | 0.040 | 0.625 | 0.040 |
| | 2 | 0.096 | 0.041 | 0.310 | 0.042 |
| | 1 | 0.063 | 0.041 | 0.169 | 0.042 |
| MEB | 0 | 0.046 | 0.042 | 0.040 | 0.041 |
| Neg. | 0 | 0.042 | 0.042 | 0.040 | 0.042 |
| control | | | | | |

The results show that the assay can detect both antigens regardless of whether the TMB reaction is performed before the alkaline phosphatase reaction or the alkaline phosphatase reaction is performed before the TMB reaction. However, the results suggests that for at least these antigens and this combination of chromogenic substrates, it is preferable to perform the alkaline phosphatase reaction before the TMB reaction.

### EXAMPLE 2

In this example various chromogenic substrates were compared in the multiplex ELISA for detecting BtCry3Bb1 (3Bb1) and BtCry1Ab/1Ac (1Ab/1Ac) in a sample.

A mixture of polyclonal antibodies against 3Bb1 and 1Ab/1Ac at a ratio of 2 to 1.5 were each coated to the bottom and sides of the wells of an ELISA plate, plates A and B respectively, using standard methods for coating ELISA plate wells with antibodies. The antibodies are available from Agdia, Inc., Elkhart, Ind.

A sample containing 3Bb1 (C1133) in MEB buffer and a sample containing 1Ab/1Ac (C1087) in MEB buffer were each serially diluted 1:8, 1:16, and 1:32. Each dilution was added to 12 wells of the above ELISA plates prepared as above to produce 12 replicates of each dilution (100 μL/well). An MEB buffer control and a negative control was included for each replicate. The plates were then incubated at room temperature for about an hour. Afterwards, the dilutions were removed from the wells and the wells washed 6-7 times with 1×PBST at room temperature. The wells were soaked about 3 minutes in 1×PBST and then the 1×PBST was tapped out.

Next, a mixture of polyclonal antibody specific for 1Ab/1Ac and conjugated to a peroxidase and polyclonal antibody specific for 3Bb1 and conjugated to alkaline phosphatase was added to each of the wells (100 μL/well. The antibodies were at an appropriate ratio in MRS buffer. After about an hour at room temperature, the antibodies were removed and the wells washed 6-7 times with 1×PBST. The wells were soaked about 3 minutes in 1×PBST and then the 1×PBST was tapped out.

Next, to replicate 1, a solution of TMB was added according to the manufacturer's instructions (100 μL/well) and after 20-30 minutes at room temperature, the absorbance at 650 nm was read using an ELISA reader. To replicate 2, a solution of BP was added according to the manufacturer's instructions (200 μL/well) and after 30-60 minutes at room temperature, the absorbance at 630 nm was read using an ELISA reader. Afterwards, the wells were washed 6-7 times with 1×PBST. The wells were soaked about 3 minutes in 1×PBST and then the 1×PBST was decanted. Then, to replicate 1, a solution of BP was added (100 μL/well) and after 30-60 minutes at room temperature, the absorbance at 630 nm was read using an ELISA reader. To replicate 2, a solution of TMB was added (100 μL/well) and after 20-30 minutes at room temperature, the absorbance at 650 nm was read using an ELISA reader. The results are shown in Table 8.

To replicate 3, a solution of TMB (Agdia, Inc.) was added according to the manufacturer's instructions (100 μL/well) and after 20-30 minutes at room temperature, the absorbance at 650 nm was read using an ELISA reader. To replicate 4, a solution of PNP (para-nitrophenyl phosphate; Agdia, Inc.) was added according to the manufacturer's instructions (100 μL/well) and after 30 minutes at room temperature, the absorbance at 405 nm was read using an ELISA reader. Afterwards, the wells were washed 6-7 times with 1×PBST. The wells were soaked about 3 minutes in 1×PBST and then the 1×PBST was decanted. Then, to replicate 3, a solution of PNP was added (100 μL/well) and after 20 minutes at room temperature, the absorbance at 405 nm was read using an ELISA reader. To replicate 4, a solution of TMB was added (100 μL/well) and after 30 minutes at room temperature, the absorbance at 650 nm was read using an ELISA reader. The results are shown in Table 8.

To replicate 5, a solution of ABTS (2,2′-azinobis-(3-ethyl-benzothiazoline-6-sulfonic acid); Agdia, Inc.) was added according to the manufacturer's instructions (100 μL/well) and after 30 minutes at room temperature, the absorbance at 405 nm was read using an ELISA reader. To replicate 6, a solution of BP was added according to the manufacturer's instructions (100 μL/well) and after 30 minutes at room temperature, the absorbance at 630 nm was read using an ELISA reader. Afterwards, the wells were washed 6-7 times with 1×PBST. The wells were soaked about 3 minutes in 1×PBST and then the 1×PBST was decanted. Then, to replicate 5, a solution of BP was added (100 μL/well) and after 20 minutes at room temperature, the absorbance at 630 nm was read using an ELISA reader. To replicate 6, a solution of ABTS was added (100 μL/well) and after 30 minutes at room temperature, the absorbance at 405 nm was read using an ELISA reader. The results are shown in Table 9.

To replicate 7, a solution of ABTS (Agdia, Inc.) was added according to the manufacturer's instructions (100 μL/well) and after 30 minutes at room temperature, the absorbance at 405 nm was read using an ELISA reader. To replicate 8, a solution of PNP was added according to the manufacturer's instructions (100 μL/well) and after 30 minutes at room temperature, the absorbance at 405 nm was read using an ELISA reader. Afterwards, the wells were washed 6-7 times with 1×PBST. The wells were soaked about 3 minutes in 1×PBST and then the 1×PBST was decanted. Then, to replicate 7, a solution of PNP was added (100 μL/well) and after 20 minutes at room temperature, the absorbance at 405 nm was read using an ELISA reader. To replicate 8, a solution of ABTS was added (100 μL/well) and after 30 minutes at room temperature, the absorbance at 405 nm was read using an ELISA reader. The results are shown in Table 9.

To replicate 9, a solution of OPD (Agdia, Inc.) was added according to the manufacturer's instructions (100 μL/well) and after 30 minutes at room temperature, the absorbance at 490 nm was read using an ELISA reader. To replicate 10, a solution of BP was added according to the manufacturer's instructions (100 μL/well) and after 30 minutes at room temperature, the absorbance at 630 nm was read using an ELISA reader. Afterwards, the wells were washed 6-7 times with 1×PBST. The wells were soaked about 3 minutes in 1×PBST and then the 1×PBST was decanted. Then, to replicate 9, a solution of BP was added (100 μL/well) and after 20 minutes at room temperature, the absorbance at 630 nm was read using an ELISA reader. To replicate 10, a solution of OPD was added (100 μL/well) and after 30 minutes at room temperature, the absorbance at 405 nm was read using an ELISA reader. The results are shown in Table 10.

To replicate 11, a solution of OPD (Agdia, Inc.) was added according to the manufacturer's instructions (100 μL/well) and after 30 minutes at room temperature, the absorbance at 490 nm was read using an ELISA reader. To replicate 12, a solution of PNP was added according to the manufacturer's instructions (100 μL/well) and after 30 minutes at room temperature, the absorbance at 405 nm was read using an ELISA reader. Afterwards, the wells were washed 6-7 times with 1×PBST. The wells were soaked about 3 minutes in 1×PBST and then the 1×PBST was decanted. Then, to replicate 11, a solution of PNP was added (100 μL/well) and after 20 minutes at room temperature, the absorbance at 405 nm was read using an ELISA reader. To replicate 12, a solution of OPD was added (100 μL/well) and after 30 minutes at room temperature, the absorbance at 490 nm was read using an ELISA reader. The results are shown in Table 10.

TABLE 8

| | | Absorbance read at 650 nm | | | |
|---|---|---|---|---|---|
| | | TMB 1^{st} | TMB 2^{nd} | TMB 1^{st} | TMB 2^{nd} |
| Sample | Dilution | 1 | 2 | 3 | 4 |
| 3Bb1 | 32 | 0.020 | 0.039 | 0.024 | 0.014 |
| | 16 | 0.021 | 0.030 | 0.021 | 0.013 |
| | 8 | 0.019 | 0.021 | 0.028 | 0.014 |
| 1Ab/1Ac | 32 | 2.784 | 2.483 | 2.746 | 2.378 |
| | 16 | 2.621 | 2.163 | 2.608 | 2.055 |
| | 8 | 2.066 | 1.561 | 2.030 | 1.462 |
| MEB | 0 | 0.024 | 0.024 | 0.025 | 0.013 |
| Neg. | 0 | 0.032 | 0.033 | 0.023 | 0.015 |
| Control | | | | | |
| | | Absorbance read at | | Absorbance read at | |
| | | 630 nm | | 405 nm | |
| | | BP 2^{nd} | BP 1^{st} | PNP 2^{nd} | PNP 1^{st} |
| Sample | Dilution | 1 | 2 | 3 | 4 |
| 3Bb1 | 32 | 1.704 | 2.617 | 1.473 | 3.000 |
| | 16 | 1.109 | 2.342 | 0.832 | 3.000 |
| | 8 | 0.451 | 1.966 | 0.388 | 2.079 |
| 1Ab/1Ac | 32 | 0.044 | 0.049 | 0.009 | 0.009 |
| | 16 | 0.045 | 0.045 | 0.010 | 0.008 |
| | 8 | 0.046 | 0.047 | 0.012 | 0.011 |
| MEB | 0 | 0.048 | 0.051 | 0.012 | 0.011 |
| Neg. | 0 | 0.060 | 0.055 | 0.013 | 0.015 |
| Control | | | | | |

TABLE 9

| | | Absorbance read at 405 nm | | | |
|---|---|---|---|---|---|
| | | ABTS 1^{st} | ABTS 2^{nd} | ABTS 1^{st} | ABTS 2^{nd} |
| Sample | Dilution | 5 | 6 | 7 | 8 |
| 3Bb1 | 32 | 0.055 | 0.048 | 0.056 | 0.052 |
| | 16 | 0.056 | 0.051 | 0.052 | 0.054 |
| | 8 | 0.051 | 0.046 | 0.053 | 0.052 |
| 1Ab/1Ac | 32 | 1.135 | 0.722 | 1.133 | 0.609 |
| | 16 | 0.842 | 0.524 | 0.813 | 0.465 |
| | 8 | 0.465 | 0.284 | 0.428 | 0.259 |
| MEB | 0 | 0.057 | 0.060 | 0.058 | 0.057 |
| Neg. | 0 | 0.060 | 0.062 | 0.060 | 0.059 |
| Control | | | | | |
| | | Absorbance read at | | Absorbance read at | |
| | | 630 nm | | 405 nm | |
| | | BP 2^{nd} | BP 1^{st} | PNP 2^{nd} | PNP 1^{st} |
| Sample | Dilution | 5 | 5 | 7 | 8 |
| 3Bb1 | 32 | 0.593 | 2.655 | 0.415 | 3.000 |
| | 16 | 0.300 | 2.315 | 0.244 | 3.000 |
| | 8 | 0.193 | 1.920 | 0.163 | 1.855 |
| 1Ab/1Ac | 32 | 0.045 | 0.041 | 0.005 | 0.006 |
| | 16 | 0.046 | 0.041 | 0.007 | 0.005 |
| | 8 | 0.045 | 0.042 | 0.007 | 0.006 |
| MEB | 0 | 0.044 | 0.043 | 0.008 | 0.010 |
| Neg. | 0 | 0.046 | 0.044 | 0.011 | 0.004 |
| Control | | | | | |

TABLE 10

| | | Absorbance read at 405 nm | | | |
|---|---|---|---|---|---|
| | | OPD 1^{st} | OPD 2^{nd} | OPD 1^{st} | OPD 2^{nd} |
| Sample | Dilution | 9 | 10 | 11 | 12 |
| 3Bb1 | 32 | 0.009 | −0.021 | 0.008 | 0.011 |
| | 16 | 0.009 | −0.011 | 0.006 | 0.010 |
| | 8 | 0.008 | −0.001 | 0.007 | 0.010 |
| 1Ab/1Ac | 32 | 3.000 | 2.681 | 3.000 | 2.637 |
| | 16 | 3.000 | 2.065 | 3.000 | 2.011 |
| | 8 | 2.190 | 1.088 | 2.174 | 1.012 |
| MEB | 0 | 0.010 | 0.008 | 0.008 | 0.010 |
| Neg. | 0 | 0.009 | 0.009 | 0.008 | 0.011 |
| Control | | | | | |
| | | Absorbance read at | | Absorbance read at | |
| | | 630 nm | | 405 nm | |
| | | BP 2^{nd} | BP 1^{st} | PNP 2^{nd} | PNP 1^{st} |
| Sample | Dilution | 9 | 10 | 11 | 12 |
| 3Bb1 | 32 | 0.042 | 2.630 | 0.002 | 3.000 |
| | 16 | 0.046 | 2.383 | 0.002 | 3.000 |
| | 8 | 0.042 | 1.904 | 0.002 | 1.861 |
| 1Ab/1Ac | 32 | 0.044 | 0.041 | 0.004 | 0.008 |
| | 16 | 0.040 | 0.040 | 0.003 | 0.008 |
| | 8 | 0.042 | 0.042 | 0.003 | 0.009 |
| MEB | 0 | 0.042 | 0.040 | 0.003 | 0.008 |
| Neg. | 0 | 0.042 | 0.042 | 0.002 | 0.008 |
| Control | | | | | |

### EXAMPLE 3

This example illustrates a multiplex ELISA for detecting BtCry2A (2A) and CP4 EPSPS(CP4) in a sample.

A mixture of polyclonal antibodies against 2A and CP4 at a ratio of 2.5 to 2 and a mixture at a ratio of 1.25 to 1 were each separately coated to the bottom and sides of the wells of ELISA plates using standard methods for coating ELISA plate wells with antibodies. The antibodies are available from Agdia, Inc., Elkhart, Ind.

The samples consisted of (1) a mixture of 2A+CP4 in PBS and TWEEN (PBST), (2) 2A, (3) CP4, (4) 1% 2A, (5) 1% CP4, (5) negative control, (6) negative control, and (7) PBST buffer. 2A and CP4 are available from Agdia, Inc. Each sample was added to four wells of each of the above ELISA plates prepared as above to produce replicates of each for each plate with 100 μL sample per well. The plates were then incubated at room temperature for about an hour. Afterwards, the samples were removed from the wells and the wells washed 6-7 times with 1×PBST. The wells were soaked about 3 minutes in 1×PBST and then the 1×PBST was decanted.

Next, a mixture of polyclonal antibody specific for 2A and conjugated to alkaline phosphatase and polyclonal antibody specific for CP4 and conjugated to peroxidase was added to each of the wells 1-4 (100 μL/well); and, a mixture of polyclonal antibody specific for 2A and conjugated to peroxidase and polyclonal antibody specific for CP4 and conjugated to alkaline phosphatase was added to each of the wells 5-8 (100 μL/well). The antibodies were at a 1:1 ratio in MRS buffer. After about an hour at room temperature, the antibodies were removed and the wells washed 6-7 times with 1×PBST. The wells were soaked about 3 minutes in 1×PBST and then the 1×PBST was decanted.

Next, to wells 1, 3, 5, and 7, a solution of TMB (Agdia, Inc.) was added according to the manufacturer's instructions (100 μL/well) and after 20-30 minutes at room temperature, the absorbance at 650 nm was read using an ELISA reader. To wells 2, 4, 6, and 8, a solution of BP was added according to the manufacturer's instructions (100 μL/well) and after 30-60 minutes at room temperature, the absorbance at 630 nm was read using an ELISA reader. Afterwards, all the wells were washed 6-7 times with 1×PBST. The wells were soaked about 3 minutes in 1×PBST and then the 1×PBST was tapped out.

Then, to wells 1, 3, 5, and 7, a solution of BP was added (100 μL/well) and after 30-60 minutes at room temperature, the absorbance at 630 nm was read using an ELISA reader. To wells 2, 4, 6, and 8, a solution of TMB was added (100 μL/well) and after 20-30 minutes at room temperature; the absorbance at 650 nm was read using an ELISA reader. The results are shown in Tables 11 and 12. For columns 1, 2, 5, and 6, the ratio of the antibodies immobilized to the wells was 2.5 to 2. For columns 3, 4, 7, and 8, the ratio of the antibodies immobilized to the wells was 1.25 to 1.

TABLE 11
Antibodies: anti-BtCry2A (AP) and anti-CP4
(peroxidase)

Absorbance read at 650 nm

| | TMB 1^{st} | TMB 2^{nd} | TMB 1^{st} | TMB 2^{nd} |
|---|---|---|---|---|
| Sample | 1 | 2 | 3 | 4 |
| 2A + CP4 | 2.825 | 1.327 | 2.804 | 1.301 |
| 2A | 1.743 | 0.389 | 1.490 | 0.359 |
| CP4 | 2.825 | 1.790 | 2.765 | 1.613 |
| 1% 2A | 0.235 | 0.181 | 0.335 | 0.226 |
| 1% CP4 | 2.649 | 1.204 | 2.483 | 1.073 |
| Neg. | 0.256 | 0.186 | 0.316 | 0.165 |
| control | | | | |
| Neg. | 0.219 | 0.134 | 0.221 | 0.245 |
| Control | | | | |
| Buffer | 0.946 | 1.099 | 1.196 | 0.975 |

Absorbance read at 630 nm

| | BP 2^{nd} | BP 1^{st} | PNP 2^{nd} | PNP 1^{st} |
|---|---|---|---|---|
| Sample | 1 | 2 | 3 | 4 |
| 2A + CP4 | 0.090 | 0.181 | 0.093 | 0.216 |
| 2A | 0.115 | 0.238 | 0.124 | 0.262 |
| CP4 | 0.044 | 0.013 | 0.047 | 0.049 |
| 1% 2A | 0.062 | 0.068 | 0.061 | 0.101 |
| 1% CP4 | 0.044 | 0.020 | 0.049 | 0.047 |
| Neg. | 0.046 | 0.015 | 0.046 | 0.046 |
| control | | | | |
| Neg. | 0.049 | 0.015 | 0.051 | 0.051 |
| Control | | | | |
| Buffer | 0.050 | 0.027 | 0.049 | 0.053 |

TABLE 12
Antibodies: anti-BtCry2A (peroxidase) and anti-CP4
(AP)

Absorbance read at 650 nm

| | TMB 1^{st} | TMB 2^{nd} | TMB 1^{st} | TMB 2^{nd} |
|---|---|---|---|---|
| Sample | 5 | 6 | 7 | 8 |
| 2A + CP4 | 1.365 | 0.977 | 1.428 | 0.950 |
| 2A | 1.715 | 1.238 | 1.723 | 1.239 |
| CP4 | 0.119 | 0.081 | 0.138 | 0.073 |
| 1% 2A | 0.636 | 0.449 | 0.623 | 0.436 |
| 1% CP4 | 0.136 | 0.081 | 0.133 | 0.085 |
| Neg. | 0.121 | 0.065 | 0.159 | 0.085 |
| control | | | | |
| Neg. | 0.134 | 0.078 | 0.140 | 0.078 |
| Control | | | | |
| Buffer | 0.143 | 0.135 | 0.299 | 0.115 |

Absorbance read at 630 nm

| | BP 2^{nd} | BP 1^{st} | PNP 2^{nd} | PNP 1^{st} |
|---|---|---|---|---|
| Sample | 5 | 6 | 7 | 8 |
| 2A + CP4 | 0.367 | 1.753 | 0.347 | 1.792 |
| 2A | 0.112 | 0.778 | 0.114 | 0.799 |
| CP4 | 0.506 | 1.865 | 0.393 | 1.850 |
| 1% 2A | 0.046 | 0.061 | 0.046 | 0.070 |
| 1% CP4 | 0.295 | 1.743 | 0.263 | 1.644 |
| Neg. | 0.042 | 0.040 | 0.044 | 0.045 |
| control | | | | |
| Neg. | 0.046 | 0.044 | 0.044 | 0.047 |
| Control | | | | |
| Buffer | 0.045 | 0.048 | 0.047 | 0.054 |

Data from Example 3 (Tables 11 and 12) also indicate the enzyme conjugate efficiency of particular anti-analyte antibody. For example the Table 11 shows the reactivity of BtCry2A alkalinephosphatase conjugate compared to BtCry2A peroxidase conjugate reactivity as shown in Table 12. In other words, the test would not perform satisfactorily if you use anti-BtCry2A alkphos conjugated antibody (high non-specific reaction). This has nothing to do with the art of the test; it is just reactivity of a particular preparation of conjugate.

### EXAMPLE 4

Mixed 1% 2A and 1% CP4 cotton seed powder equally for mixed sample and compared the signals from the mixture in the multiplex ELISA to the signal produced by 2A and CP4 controls. The results are shown in Tables 13 and 14. In the Tables, columns 1 and 4 are 2A controls, columns 2 and 5 are CP4 controls, and columns 3 and 6 are the above 2A and CP4 cotton seed mixtures.

The antibodies against the 2A were conjugated to alkaline phosphatase and the antibodies against the CP4 were conjugated to peroxidase. The substrate for the alkaline phosphatase was BP and the substrate for the peroxidase was TMB. The ELISA was performed as above. For all samples, the alkaline phosphatase assay was performed first and the peroxidase assay was performed second.

For columns 1, 2, and 3, the ratio of the antibodies immobilized to the wells was 2.5 to 2. For columns 4, 5, and 6, the ratio of the antibodies immobilized to the wells was 1.25 to 1.

TABLE 13

| | | Absorbance read at 630 nm (BP) | | |
|---|---|---|---|---|
| | | 2A | CP4 | 2A + CP4 |
| Sample | Dilution | 1 | 2 | 3 |
| 1% seed | 10 | 0.243 | 0.039 | 0.172 |
| | 20 | 0.229 | 0.042 | 0.139 |
| | 40 | 0.172 | 0.041 | 0.126 |
| | 80 | 0.143 | 0.047 | 0.100 |
| | 160 | 0.096 | 0.043 | 0.081 |
| | 320 | 0.090 | 0.052 | 0.073 |
| MEB | 0 | 0.057 | 0.044 | 0.061 |
| Neg. | 0 | 0.055 | 0.043 | 0.058 |
| Control | | | | |
| | | Absorbance read at 650 nm (TMB) | | |
| | | 2A | CP4 | 2A + CP4 |
| Sample | Dilution | 1 | 2 | 3 |
| 1% seed | 10 | 0.004 | 0.781 | 0.533 |
| | 20 | 0.004 | 0.598 | 0.282 |
| | 40 | 0.001 | 0.314 | 0.127 |
| | 80 | 0.000 | 0.147 | 0.165 |
| | 160 | 0.001 | 0.059 | 0.037 |
| | 320 | 0.000 | 0.021 | 0.017 |
| MEB | 0 | 0.000 | −0.011 | 0.005 |
| Neg. | 0 | 0.003 | −0.008 | 0.007 |
| Control | | | | |

TABLE 14

| | | Absorbance read at 630 nm (BP) | | |
|---|---|---|---|---|
| | | 2A | CP4 | 2A + CP4 |
| Sample | Dilution | 4 | 5 | 6 |
| 1% seed | 10 | 0.256 | 0.068 | 0.153 |
| | 20 | 0.281 | 0.053 | 0.140 |
| | 40 | 0.175 | 0.055 | 0.126 |
| | 80 | 0.129 | 0.058 | 0.102 |
| | 160 | 0.105 | 0.060 | 0.080 |
| | 320 | 0.097 | 0.058 | 0.075 |
| MEB | 0 | 0.060 | 0.059 | 0.055 |
| Neg. | 0 | 0.052 | 0.059 | 0.057 |
| Control | | | | |
| | | Absorbance read at 650 nm (TMB) | | |
| | | 2A | CP4 | 2A + CP4 |
| Sample | Dilution | 4 | 5 | 6 |
| 1% seed | 10 | 0.006 | 0.742 | 0.390 |
| | 20 | 0.005 | 0.581 | 0.275 |
| | 40 | 0.005 | 0.343 | 0.124 |
| | 80 | 0.003 | 0.133 | 0.106 |
| | 160 | 0.003 | 0.062 | 0.028 |
| | 320 | 0.004 | 0.036 | 0.020 |
| MEB | 0 | 0.002 | 0.005 | 0.003 |
| Neg. | 0 | 0.003 | 0.003 | 0.003 |
| Control | | | | |

### EXAMPLE 5

Mixed 1% 2A and 1% CP4 cotton seed powder equally for mixed sample and compared the signals from the mixture in the multiplex ELISA to the signal produced by 2A and CP4 controls. The results are shown in Tables 15 and 16. In the Tables, columns 1 and 4 are 2A controls, columns 2 and 5 are CP4 controls, and columns 3 and 6 are the above 2A and CP4 cotton seed mixtures.

The antibodies against the 2A were conjugated to alkaline phosphatase and the antibodies against the CP4 were conjugated to peroxidase. The substrate for the alkaline phosphatase was pNP and the substrate for the peroxidase was TMB. The ELISA was performed as above. For all samples, the peroxidase assay was performed first and the alkaline phosphatase assay was performed second.

For columns 1, 2, and 3, the ratio of the antibodies immobilized to the wells was 2.5 to 2. For columns 4, 5, and 6, the ratio of the antibodies immobilized to the wells was 1.25 to 1.

TABLE 15

| | | Absorbance read at 650 nm (TMB) | | |
|---|---|---|---|---|
| | | 2A | CP4 | 2A + CP4 |
| Sample | Dilution | 1 | 2 | 3 |
| 1% seed | 10 | 0.100 | 2.234 | 1.950 |
| | 20 | 0.066 | 2.073 | 1.713 |
| | 40 | 0.033 | 1.723 | 1.131 |
| | 80 | 0.023 | 1.101 | 0.609 |
| | 160 | 0.012 | 0.623 | 0.336 |
| | 320 | 0.012 | 0.324 | 0.195 |
| MEB | 0 | 0.008 | −0.003 | 0.012 |
| Neg. | 0 | 0.011 | 0.003 | 0.012 |
| Control | | | | |
| | | Absorbance read at 405 nm (PNP) | | |
| | | 2A | CP4 | 2A + CP4 |
| Sample | Dilution | 1 | 2 | 3 |
| 1% seed | 10 | 0.104 | 0.013 | 0.059 |
| | 20 | 0.096 | 0.016 | 0.058 |
| | 40 | 0.077 | 0.017 | 0.047 |
| | 80 | 0.053 | 0.019 | 0.035 |
| | 160 | 0.037 | 0.020 | 0.033 |
| | 320 | 0.027 | 0.021 | 0.024 |
| MEB | 0 | 0.019 | 0.022 | 0.020 |
| Neg. | 0 | 0.013 | 0.015 | 0.012 |
| Control | | | | |

This Example shows that PNP should be added first to avoid interference by TMB.

TABLE 16

| | | Absorbance read at 650 nm (TMB) | | |
|---|---|---|---|---|
| | | 2A | CP4 | 2A + CP4 |
| Sample | Dilution | 4 | 5 | 6 |
| 1% seed | 10 | 0.104 | 2.153 | 1.942 |
| | 20 | 0.076 | 2.023 | 1.658 |
| | 40 | 0.033 | 1.631 | 1.129 |
| | 80 | 0.020 | 1.123 | 0.644 |
| | 160 | 0.015 | 0.634 | 0.373 |
| | 320 | 0.009 | 0.361 | 0.183 |
| MEB | 0 | 0.007 | 0.007 | 0.008 |
| Neg. | 0 | 0.008 | 0.010 | 0.009 |
| Control | | | | |
| | | Absorbance read at 405 nm (PNP) | | |
| | | 2A | CP4 | 2A + CP4 |
| Sample | Dilution | 4 | 5 | 6 |
| 1% seed | 10 | 0.099 | 0.012 | 0.058 |
| | 20 | 0.090 | 0.013 | 0.052 |
| | 40 | 0.068 | 0.016 | 0.048 |
| | 80 | 0.050 | 0.017 | 0.034 |
| | 160 | 0.037 | 0.019 | 0.039 |
| | 320 | 0.033 | 0.019 | 0.025 |
| MEB | 0 | 0.017 | 0.018 | 0.019 |
| Neg. | 0 | 0.012 | 0.014 | 0.011 |
| Control | | | | |

### EXAMPLE 6

A typical protocol for performing the multiplex ELISA using an equilibrium protocol is as follows.

Add 100 μL of a mixture of antibody conjugates to the well of an ELISA plate containing immobilized antibodies. There is a conjugate species for each analyte to be detected in a sample. Then add 100 μL of sample or control to the wells. Incubate the samples for about an hour, then remove mixture and sample from the wells and wash the wells as in the previous examples. Then add the first chromogenic substrate, incubate for an appropriate time, read optical density (OD), wash wells, add next chromogenic substrate, incubate for an appropriate time, read OD, wash wells, and repeat for each analyte to be detected. For example, add 100 μL of PNP substrate solution, incubate 30 minutes, and then read the OD at 405 nm, wash wells, add 100 μL H₂O₂/TMB, incubate 30 minutes, and then read OD at 650 nm.

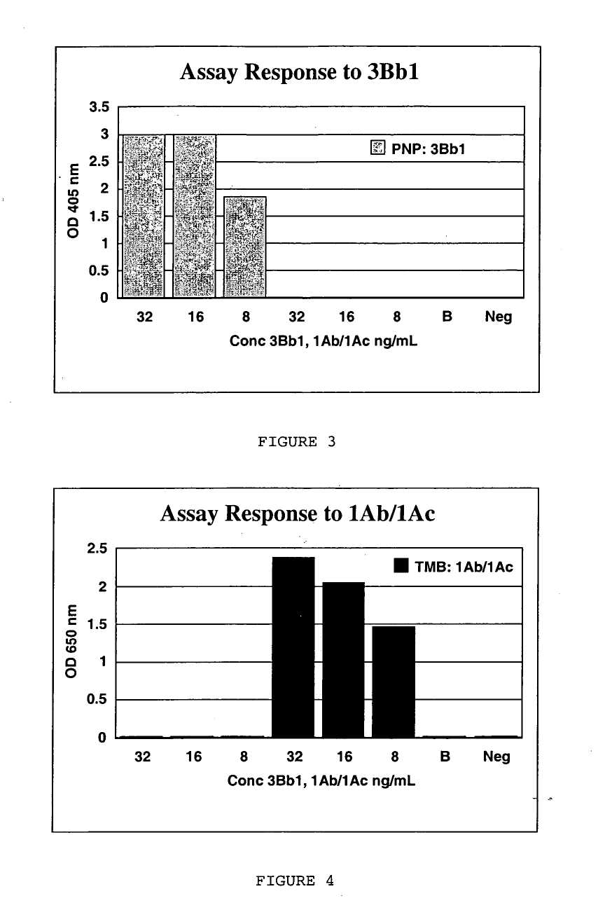

The results of an equilibrium assay for detecting 3Bb1 and 1Ab/1Ac is shown in Table 17 and 18 and FIGS. 3 and 4.

TABLE 17
PNP Read First

| Sample | Concentration | 405 nm |
|---|---|---|
| 3Bb1 | 32 | 3.00 |
| 3Bb1 | 16 | 3.00 |
| 3Bb1 | 8 | 1.855 |
| 1Ab/1Ac | 32 | 0.006 |
| 1Ab/1Ac | 16 | 0.005 |
| 1Ab/1Ac | 8 | 0.006 |
| Buffer | — | 0.010 |
| Neg. control | — | 0.004 |

TABLE 18
TMB Read Second

| Sample | Concentration | 650 nm |
|---|---|---|
| 3Bb1 | 32 | 0.014 |
| 3Bb1 | 16 | 0.013 |
| 3Bb1 | 8 | 0.014 |
| 1Ab/1Ac | 32 | 2.378 |
| 1Ab/1Ac | 16 | 2.055 |
| 1Ab/1Ac | 8 | 1.462 |
| Buffer | — | 0.013 |
| Neg. control | — | 0.015 |

In conventional ELISAs to detect two analytes, two plates or sets of wells are needed and there are 6 pipetting steps and two washes to detect two analytes. In the equilibrium multiplex ELISA of the present invention, there are 4 pipetting steps and two washes to detect two analytes. Thus, the present invention reduces the chances for pipetting errors. The present invention uses less samples and reagents, uses less space, is less costly, and is less complex than conventional ELISAs.

The following are further examples which illustrate particular embodiments of the multiplex ELISA of the present invention.

### EXAMPLE 7

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2/1 μg/mL was coated to the bottom of the wells of ELISA plates using standard methods for coating ELISA plate wells with antibodies. The antibodies are available from Agdia, Inc., Elkhart, Ind.

The samples consisted of (1) positive control for 3Bb1 at 32 ng/mL in Milk Extraction Buffer (MEB), (2) positive control for 3Bb1 at 16 ng/mL in MEB, (3) positive control for 3Bb1 at 8 ng/mL in MEB, (4) positive control for 1Ab at 32 ng/mL in MEB, (5) positive control for 1Ab at 16 ng/mL in MEB, (6) positive control for 1Ab at 8 ng/ml in MEB, (7) MEB, and (8) negative control. 3Bb1 and 1Ab are available from Agdia, Inc. Each sample was added to five wells of the above ELISA plate prepared as above with 100 μL sample per well. The plates were then incubated at room temperature for about an hour. Afterwards, the samples were removed from the wells and the wells washed 6-7 times with 1×PBST. The wells were soaked for 3 minutes in 1×PBST was tapped out.

Next, a mixture of monoclonal antibody specific for 3Bb1 and conjugated to alkaline phosphatase and polyclonal antibody specific for 1Ab and conjugated to peroxidase was added to each of the wells (100 μL/well). The antibodies were at a 1:1 ratio in MRS buffer. After an hour at room temperature, the antibodies were removed and the wells washed 6-7 times with 1×PBST. The wells were soaked for 3 minutes in 1×PBST and then emptied.

Next, a solution of PNP was added (100 μL/well) and after 30-60 minutes at room temperature, the absorbance at 405 nm was read using an ELISA reader. After reading at 405 nm, the substrate was removed and the wells, washed 4-5 times with 1×PBST. Then a solution of TMB was added (100 μL/well) and after 20 minutes at room temperature, the absorbance at 650 nm was read using an ELISA reader. The results are shown in Tables 19 and 20.

TABLE 19
Absorbance read at 405 nm

| | 1 |
|---|---|
| 3Bb1 - 32 | 3.000 |
| 3Bb1 - 16 | 2.832 |
| 3Bb1 - 8 | 1.789 |
| 1Ab - 32 | 0.005 |
| 1Ab - 16 | 0.007 |
| 1Ab - 8 | 0.007 |
| Buffer | 0.009 |
| Neg. | 0.010 |
| Control | |

TABLE 20
Absorbance read at 650 nm

| | 1 |
|---|---|
| 3Bb1 - 32 | 0.010 |
| 3Bb1 - 16 | 0.018 |
| 3Bb1 - 8 | 0.015 |
| 1Ab - 32 | 1.522 |
| 1Ab - 16 | 1.316 |
| 1Ab - 8 | 0.952 |
| Buffer | 0.015 |
| Neg. | 0.015 |
| Control | |

### EXAMPLE 8

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2/1 μg/mL was coated to the bottom of the wells of ELISA plates using standard methods for coating ELISA plate wells with antibodies. The antibodies are available from Agdia, Inc., Elkhart, Ind.

The samples consisted of (1) positive control for 3Bb1 at 32 ng/mL in Milk Extraction Buffer (MEB), (2) positive control of 3Bb1 at 10.7 ng/mL, (3) positive control for 3Bb1 at 3.6 ng/mL, (4) positive control for 1Ab at 32 ng/mL, (5) positive control for 1Ab at 10.7 ng/mL, (6) positive control for 1Ab at 3.6 ng/ml, (7) MEB, and (8) negative control. 3Bb1 and 1Ab are available from Agdia, Inc. Each sample was added to five wells of the above ELISA plate prepared as above with 100 μL sample per well. The plates were then incubated at room temperature for about an hour. Afterwards, the samples were removed from the wells and the wells washed 6-7 times with 1×PBST. The wells were soaked for 3 minutes in 1×PBST and then decanted.

Next, a mixture of monoclonal antibody specific for 3Bb1 and conjugated to alkaline phosphatase and polyclonal antibody specific for 1Ab and conjugated to peroxidase was added to each of the wells (100 μL/well). The antibodies were at ratio of 1:1 in MRS buffer. After about an hour at room temperature, the antibodies were removed and the wells washed 6-7 times with 1×PBST. The wells were soaked about 3 minutes in 1×PBST and then decanted.

Next, a solution of PNP was added (100 μL/well) and after 30-60 minutes at room temperature, the absorbance at 405 nm was read using an ELISA reader. After reading at 405 nm, the substrate was removed and the wells washed 4-5 times with 1×PBST. Then a solution of TMB was added (100 μL/well) and after 20 minutes at room temperature, the absorbance at 650 nm was read using an ELISA reader. The results are shown in Tables 21 and 22.

TABLE 21
Absorbance read at 405 nm

| | 1 |
|---|---|
| 3Bb1 - 32 | 3.000 |
| 3Bb1 - 10.7 | 2.149 |
| 3Bb1 - 3.6 | 0.770 |
| 1Ab - 32 | 0.012 |
| 1Ab - 10.7 | 0.013 |
| 1Ab - 3.6 | 0.014 |
| Buffer | 0.016 |
| Neg. Control | 0.014 |

TABLE 22
Absorbance read at 650 nm

| | 1 |
|---|---|
| 3Bb1 - 32 | 0.019 |
| 3Bb1 - 10.7 | 0.014 |
| 3Bb1 - 3.6 | 0.011 |
| 1Ab - 32 | 2.187 |
| 1Ab - 10.7 | 1.655 |
| 1Ab - 3.6 | 0.575 |
| Buffer | 0.017 |
| Neg. Control | 0.020 |

### EXAMPLE 9

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2 to 1 was coated to the bottom of the wells of ELISA plates using standard methods for coating ELISA plate wells with antibodies. The antibodies are available from Agdia, Inc., Elkhart, Ind.

The samples consisted of (1) positive control for 3Bb1 at 32 ng/mL in Milk Extraction Buffer (MEB), (2) positive control for 3Bb1 at 10.7 ng/mL, (3) positive control for 3Bb1 at 3.6 ng/mL, (4) positive control for 1Ab at 32 ng/mL, (5) positive control for 1Ab at 10.7 ng/mL, (6) positive control for 1Ab at 3.6 ng/ml, (7) MEB, and (8) negative control. 3Bb1 and 1Ab are available from Agdia, Inc. Each sample was added to four wells of the above ELISA plate prepared as above with 100 μL sample per well. The plates were then incubated at room temperature for about an hour. Afterwards, the samples were removed from the wells and the wells washed 6-7 times with 1×PBST. The wells were soaked for 3 minutes in 1×PBST and then decanted.

Next, a mixture of monoclonal antibody specific 3Bb1 and conjugated to alkaline phosphatase and polyclonal antibody specific for 1Ab and conjugated to peroxidase was added to each of the wells (100 μL/well). The antibodies were at a ratio of 1:1 in MRS buffer. After about an hour at room temperature, the antibodies were removed and the wells, washed 6-7 times with 1×PBST. The wells were soaked about 3 minutes in 1×PBST and then decanted.

Next, a solution of PNP was added (100 μL/well) and after 30-60 minutes at room temperature, the absorbance at 405 nm was read using an ELISA reader. After reading at 405 nm, the substrate was removed and the wells washed 4-5 times with 1×PBST. Then a solution of TMB was added (100 μL/well) and after 20 minutes at room temperature, the absorbance at 650 nm was read using an ELISA reader. The results are shown in Tables 23 and 24.

TABLE 23
Absorbance read at 405 nm

| | 1 |
|---|---|
| 3Bb1 - 32 | 2.859 |
| 3Bb1 - 10.7 | 1.738 |
| 3Bb1 - 3.6 | 0.847 |
| 1Ab - 32 | 0.014 |
| 1Ab - 10.7 | 0.013 |
| 1Ab - 3.6 | 0.014 |
| Buffer | 0.018 |
| Neg. Control | 0.017 |

TABLE 24
Absorbance read at 650 nm

| | 1 |
|---|---|
| 3Bb1 - 32 | 0.009 |
| 3Bb1 - 10.7 | 0.011 |
| 3Bb1 - 3.6 | 0.024 |
| 1Ab - 32 | 0.615 |
| 1Ab - 10.7 | 0.424 |
| 1Ab - 3.6 | 0.227 |
| Buffer | 0.037 |
| Neg. Control | 0.013 |

### EXAMPLE 10

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2 to 1 was coated to the bottom and sides of the wells of ELISA plates using standard methods for coating ELISA plate wells with antibodies. The antibodies are available from Agdia, Inc., Elkhart, Ind.

The samples consisted of (1) positive control for 3Bb1 at 32 ng/mL in Milk Extraction Buffer (MEB), (2) positive control for 3Bb1 at 10.7 ng/mL, (3) positive control for 3Bb1 at 3.6 ng/mL, (4) positive control for 1Ab at 32 ng/Ml, (5) positive control for 1Ab at 10.7 ng/mL, (6) positive control for 1Ab at 3.6 ng/ml, (7) MEB, and (8) negative control. 3Bb1 and 1Ab are available from Agdia, Inc. Each sample was added to four wells of the above ELISA plate prepared as above with 100 μL sample per well. The plates were then incubated at room temperature for about an hour. Afterwards, the samples were removed from the wells and the wells washed 6-7 times with 1×PBST. The wells were soaked for 3 minutes in 2×PBST and then decanted.

Next, a mixture of monoclonal antibody specific for 3Bb1 and conjugated to alkaline phosphatase and polyclonal antibody specific for 1Ab and conjugated to peroxidase was added to each of the wells (100 μL/well). The antibodies were at a 1:1 ratio in MRS buffer. After about an hour at room temperature, the antibodies were removed and the wells washed 6-7 times with 1×PBST and then decanted.

Next, a solution of Agdia's PNP was added to wells 1-16 (100 μL/well), and a solution of Ready PNP was added to wells 17-32 (100 μL/well), and after 30-60 minutes at room temperature, the absorbance at 405 nm was read using an ELISA reader. After reading at 405 nm, the substrate was removed and the wells washed 4-5 times with 1×PBST. Then a solution of Agdia's TMB was added to wells 1-8 and 17-24 (100 μL/well), and a solution of Prestained TMB was added to wells 9-16 and 25-32 (100 μL/well), and after 20 minutes at room temperature, the absorbance at 650 nm was read using an ELISA reader. Reach PNP and Prestained TMB are available from Kem-En-Tec Diagnostics, København, Denmark. The results are shown in Tables 25 and 26. For column 1, the PNP used was Agdia's PNP and the TMB used was Agdia's TMB. For column 2, the PNP used was Agdia's PNP and the TMB used was Prestained TMB. For column 3, the PNP used was Ready PNP and the TMB used was Agdia's TMB. For column 4, the PNP used was Ready PNP and the TMB used was Prestained TMB.

TABLE 25
Absorbance read at 405 nm

| | 1 | 2 | 3 | 4 |
|---|---|---|---|---|
| 3Bb1 - 32 | 3.000 | 3.000 | 2.758 | 2.706 |
| 3Bb1 - 10.7 | 2.195 | 2.067 | 1.642 | 1.523 |
| 3Bb1 - 3.6 | 0.771 | 0.760 | 0.668 | 0.568 |
| 1Ab - 32 | 0.027 | 0.024 | 0.016 | 0.016 |
| 1Ab - 10.7 | 0.026 | 0.027 | 0.016 | 0.015 |
| 1Ab - 3.6 | 0.027 | 0.027 | 0.018 | 0.016 |
| Buffer | 0.030 | 0.029 | 0.018 | 0.019 |
| Neg. Control | 0.029 | 0.029 | 0.023 | 0.020 |

TABLE 26
Absorbance read at 650 nm

| | 1 | 2 | 3 | 4 |
|---|---|---|---|---|
| 3Bb1 - 32 | 0.002 | 0.013 | 0.012 | 0.012 |
| 3Bb1 - 10.7 | 0.001 | 0.013 | 0.011 | 0.011 |
| 3Bb1 - 3.6 | 0.004 | 0.015 | 0.012 | 0.015 |
| 1Ab - 32 | 2.428 | 2.695 | 2.514 | 2.784 |
| 1Ab - 10.7 | 1.912 | 2.172 | 2.073 | 2.327 |
| 1Ab - 3.6 | 0.935 | 1.071 | 1.015 | 1.166 |
| Buffer | 0.006 | 0.015 | 0.015 | 0.014 |
| Neg. Control | 0.010 | 0.016 | 0.015 | 0.016 |

### EXAMPLE 11

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2 to 1 was coated to the bottom and sides of the wells of ELISA plates using standard methods for coating ELSA plate wells with antibodies. The antibodies are available from Agdia, Inc., Elkhart, Ind.

The samples consisted of (1) positive control for 3Bb1 at 32 ng/mL in Milk Extraction Buffer (MEB), (2) positive control for 3Bb1 at 10.7 ng/mL, (3) positive control for 3Bb1 at 3.6 ng/mL, (4) positive control for 1Ab at 32 ng/mL, (5) positive control for 1Ab at 10.7 ng/mL, (6) positive control for 1Ab at 3.6 ng/ml, (7) MEB, and (8) negative control. 3Bb1 and 1Ab are available from Agdia, Inc. Each sample was added to four wells of the above ELISA plate prepared as above with 100 μL sample per well. The plates were then incubated at room temperature for about an hour. Afterwards, the samples were removed from the wells and the wells washed 6-7 times with 1×PBST. The wells were soaked for 3 minutes in 1×PBST and then decanted.

Next, a mixture of monoclonal antibody specific for 3Bb1 and conjugated to alkaline phosphatase and polyclonal antibody specific for 1Ab and conjugated to peroxidase was added to each of the wells (100 μL/well). The antibodies were at a 1:1 ratio in MRS buffer, or at a 1:1 ratio in Dilutabody Red. After about an hour at room temperature, the antibodies were removed and the wells washed 6-7 times with 1×PBST. The wells were soaked for 3 minutes in 1×PBST and then the 1×PBST was decanted.

Next, a solution of Ready PNP was added (100 μL/well), and after 30-60 minutes at room temperature, the absorbance at 405 nm was read using an ELSA reader. After reading at 405 nm, the substrate was removed and the wells washed 4-5 times with 1×PBST. Then a solution of Prestained TMB was added (100 μL/well), and after 20 minutes at room temperature, the absorbance at 650 nm was read using an ELISA reader. Ready PNP and Prestained TMB are available from Kem-En-Tec Diagnostics, København, Denmark. The results are shown in Tables 27 and 28. For column 1, the buffer used was MRS. For column 2, the buffer used was Dilutabody red.

TABLE 27
Absorbance read at 405 nm

| | 1 | 2 |
|---|---|---|
| 3Bb1 - 32 | 3.000 | 2.318 |
| 3Bb1 - 10.7 | 1.866 | 1.335 |
| 3Bb1 - 3.6 | 0.746 | 0.531 |
| 1Ab - 32 | 0.018 | 0.016 |
| 1Ab - 10.7 | 0.019 | 0.017 |
| 1Ab - 3.6 | 0.019 | 0.018 |
| Buffer | 0.022 | 0.020 |
| Neg. Control | 0.057 | 0.021 |

TABLE 28
Absorbance read at 650 nm

| | 1 | 2 |
|---|---|---|
| 3Bb1 - 32 | 0.013 | 0.010 |
| 3Bb1 - 10.7 | 0.012 | 0.011 |
| 3Bb1 - 3.6 | 0.011 | 0.009 |
| 1Ab - 32 | 2.825 | 2.784 |
| 1Ab - 10.7 | 2.547 | 2.473 |
| 1Ab - 3.6 | 1.543 | 1.270 |
| Buffer | 0.017 | 0.012 |
| Neg. Control | 0.032 | 0.024 |

### EXAMPLE 12

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2 to 1 was coated to the bottom and sides of the wells of ELISA plates using standard methods for coating ELISA plate wells with antibodies. The antibodies are available from Agdia, Inc., Elkhart, Ind.

The samples consisted of (1) positive control for 3Bb1 or positive control for 1Ab at 32 ng/mL in Milk Extraction Buffer (MEB), (2) positive control for 3Bb1 or positive control for 1Ab at 8 ng/mL, (4) positive control for 3Bb1 or positive control for 1Ab at 4 ng/mL, (5) positive control for 3Bb1 or positive control for 1Ab at 2 ng/mL, (6) positive control for 3Bb1 or positive control for 1Ab at 1 ng/ml, (7) MEB, and (8) negative control. 3Bb1 and 1Ab are available from Agdia, Inc. Each sample was added to two wells of the above ELISA plate prepared as above with 100 μL sample per well. Wells 1-16 were then incubated at room temperature for about an hour. Afterwards, the samples were removed from the wells and the wells washed 6-7 times with 1×PBST. The wells were soaked about 3 minutes in 1×PBST and then the 1×PBST was decanted.

For wells 17-32 a mixture of monoclonal antibody specific for 3Bb1 and conjugated to alkaline phosphatase and polyclonal antibody specific for 1Ab and conjugated to peroxidase was added (100 μL/well) and the samples plus enzyme conjugates were incubated at room temperature for about 2 hours. The antibodies were at a 1:1 ratio in MRS buffer. Afterwards, the samples and enzyme conjugate were removed from the wells and the wells washed 6-7 times with 1×PBST. The wells were soaked about 3 minutes in 1×PBST and then the 1×PBST was decanted. Wells 1-16 were thus a two-step procedure, one hour incubation with sample and one hour incubation with enzyme conjugate. Wells 17-32 were a QTA procedure with a two hour incubation of sample plus enzyme conjugate.

Next, a mixture of monoclonal antibody specific for 3Bb1 and conjugated to alkaline phosphatase and polyclonal antibody specific for 1Ab and conjugated to peroxidase was added to wells 1-16 (100 μL/well). The antibodies were at a 1:1 ratio in MRS buffer. After about an hour at room temperature, the antibodies were removed and the wells washed 6-7 times with 1×PBST. The wells were soaked about 3 minutes in 1×PBST and then the 1×PBST was decanted.

Next, a solution of READY PNP was added to all wells (100 μL/well), and after 30-60 minutes at room temperature, the absorbance at 405 nm was read using an ELISA reader. After reading at 405 nm, the substrate was removed and the wells washed 4-5 times with 1×PBST. Then a solution of prestained TMB was added (100 μL/well), and after 20 minutes at room temperature, the absorbance at 650 nm was read using an ELISA reader. Ready PNP and Prestained TMB are available form Kem-En-Tec Diagnostics, København, Denmark. The results are shown in Tables 29 and 30. For column 1, the samples were a dilution of the 3Bb1 positive control and used a two step procedure. For column 2, the samples were a dilution of the 1Ab positive control and used a two step procedure. For column 3, the samples were a dilution of the 3Bb1 positive control and used a QTA procedure. For column 4, the samples were a dilution of the 1Ab positive control and used a QTA procedure.

TABLE 29
Absorbance read at 405 nm

| | 1 | 2 | 3 | 4 |
|---|---|---|---|---|
| 32 | 2.176 | 0.020 | 1.665 | 0.023 |
| 16 | 1.960 | 0.020 | 1.146 | 0.026 |
| 8 | 1.191 | 0.020 | 0.422 | 0.023 |
| 4 | 0.634 | 0.019 | 0.196 | 0.022 |
| 2 | 0.318 | 0.020 | 0.105 | 0.023 |
| 1 | 0.172 | 0.021 | 0.068 | 0.023 |
| Buffer | 0.024 | 0.021 | 0.026 | 0.022 |
| Neg. Control | 0.059 | 0.055 | 0.027 | 0.022 |

TABLE 30
Absorbance read at 650 nm

| | 1 | 2 | 3 | 4 |
|---|---|---|---|---|
| 32 | 0.010 | 2.765 | 0.008 | 2.570 |
| 16 | 0.013 | 2.547 | 0.007 | 2.378 |
| 8 | 0.009 | 2.093 | 0.008 | 1.811 |
| 4 | 0.008 | 1.538 | 0.008 | 1.202 |
| 2 | 0.009 | 0.936 | 0.009 | 0.719 |
| 1 | 0.010 | 0.512 | 0.010 | 0.405 |
| Buffer | 0.011 | 0.011 | 0.008 | 0.009 |
| Neg. Control | 0.024 | 0.022 | 0.022 | 0.021 |

### EXAMPLE 13

This example illustrates a multiplex ELISA for detecting BtCry3Bb1 (3Bb1) and BtCry1Ab (1Ab) in a sample.

A mixture of polyclonal antibodies against 3Bb1 and 1Ab at a ratio of 2 to 1 was coated to the bottom and sides of the wells of ELISA plates using standard methods for coating ELISA plate wells with antibodies. The antibodies are available from Agdia, Inc., Elkhart, Ind.

The samples consisted of (1) positive control for 3Bb1 at 16 ng/mL in Milk Extraction Buffer (MEB), (2) positive control for 3Bb1 or positive control for 1Ab at 8 ng/mL, (3) positive control for 3Bb1 or positive control for 1Ab at 4 ng/mL, (4) positive control for 3Bb1 or positive control for 1Ab at 2 ng/mL, (5) positive control for 3Bb1 or positive control for 1Ab at 1 ng/mL, (6) positive control for 3Bb1 or positive control for 1Ab at 0.5 ng/mL, (7) positive control for 1Ab at 0.25 ng/mL, (8) MEB, (9) negative control, (10) negative corn seed extracted in MEB, (11) 3Bb1 positive control at 16 ng/mL in negative corn seed extract, (12) 3Bb1 at 4 ng/mL, in negative corn seed extract, (13) 3Bb1 at 1 ng/mL in negative corn seed extract, (14) 1Ab positive control at 8 ng/mL in negative corn seed extract, (15) 1Ab positive control at 2 ng/mL in negative corn seed extract, or (16) 1Ab positive control at 0.5 ng/mL in negative corn seed extract. 3Bb1 and 1Ab are available from Agdia, Inc. Each sample was added to two wells of the above ELISA plate prepared as above with 100 μL sample per well. Wells 1-24 were then incubated at room temperature for about an hour. Afterwards, the samples were removed from the wells and the wells washed 6-7 times with 1×PBST. The wells were soaked about 3 minutes in 1×PBST and then the 1×PBST was decanted.

For wells 32-48, a mixture of monoclonal antibody specific for 3Bb1 and conjugated to alkaline phosphatase and polyclonal antibody specific for 1Ab and conjugated to peroxidase was added (100 μL/well) and the samples plus enzyme conjugates were incubated at room temperature for about 2 hours. The antibodies were at a 1:1 ratio in MRS buffer. Afterwards, the samples and enzyme conjugate were removed from the wells and the wells washed 6-7 times with 1×PBST. The wells were soaked about 3 minutes in 1×PBST and then the 1×PBST was decanted. Wells 1-16 were thus a two-step procedure, one hour incubation with sample and one hour incubation with enzyme conjugate. Wells 32-48 were a QTA procedure with a two hour incubation of sample plus enzyme conjugate.

Next, a mixture of monoclonal antibody specific for 3Bb1 and conjugated to alkaline phosphatase and polyclonal antibody specific for 1Ab and conjugated to peroxidase was added to wells 1-24 (100 μL/well). The antibodies were at a 1:1 ratio in MRS buffer. After about an hour at room temperature, the antibodies were removed and the wells washed 6-7 times with 1×PBST. The wells were soaked about 3 minutes in 1×PBST and then the 1×PBST was decanted.

Next, a solution of Ready PNP was added to all wells (100 μL/well), and after 30-60 minutes at room temperature, the absorbance at 405 nm was read using an ELSA reader. After reading at 405 nm, the substrate was removed and the wells washed 4-5 times with 1×PBST. Then a solution of Prestained TMB was added (100 μL/well), and after 20 minutes at room temperature, the absorbance at 650 nm was read using an ELSA reader. Ready PNP and Prestained TMB are available from Kem-En-Tec Diagnostics, København, Denmark. The results are shown in Tables 31 and 32. For column 1, the two step procedure was used. For column 2, the QTA procedure was used.

TABLE 31
Absorbance read at 405 nm

| | 1 | 2 |
|---|---|---|
| 3Bb1 - 16 | 2.474 | 1.084 |
| 3Bb1 - 8 | 1.820 | 0.648 |
| 3Bb1 - 4 | 1.080 | 0.238 |
| 3Bb1 - 2 | 0.606 | 0.125 |
| 3Bb1 - 1 | 0.292 | 0.070 |
| 3Bb1 - 0.5 | 0.162 | 0.044 |
| Buffer | 0.035 | 0.024 |
| Neg. Control | 0.042 | 0.029 |
| 1Ab - 8 | 0.031 | 0.026 |
| 1Ab - 4 | 0.024 | 0.024 |
| 1Ab - 2 | 0.021 | 0.024 |
| 1Ab - 1 | 0.025 | 0.025 |
| 1Ab - 0.5 | 0.025 | 0.028 |
| 1Ab - 0.2 | 0.027 | 0.027 |
| Buffer | 0.033 | 0.027 |
| Neg. Control | 0.030 | 0.026 |
| 3Bb1 - 16 in neg. seed | 1.908 | 0.873 |
| 3Bb1 - 4 in neg. seed | 0.763 | 0.288 |
| 3Bb1 - 1 in neg. seed | 0.229 | 0.080 |
| Neg. Seed | 0.027 | 0.024 |
| 1Ab - 8 in neg. seed | 0.030 | 0.021 |
| 1Ab - 2 in neg. seed | 0.032 | 0.022 |
| 1Ab - 0.5 in neg. seed | 0.032 | 0.025 |
| Neg. seed | 0.032 | 0.018 |

TABLE 32
Absorbance read at 650 nm

| | 1 | 2 |
|---|---|---|
| 3Bb1 - 16 | 0.018 | 0.020 |
| 3Bb1 - 8 | 0.006 | 0.013 |
| 3Bb1 - 4 | 0.008 | 0.014 |
| 3Bb1 - 2 | 0.006 | 0.012 |
| 3Bb1 - 1 | 0.007 | 0.012 |
| 3Bb1 - 0.5 | 0.007 | 0.015 |
| Buffer | 0.011 | 0.015 |
| Neg. Control | 0.018 | 0.013 |
| 1Ab - 8 | 2.197 | 1.828 |
| 1Ab - 4 | 1.755 | 1.326 |
| 1Ab - 2 | 0.989 | 0.677 |
| 1Ab - 1 | 0.580 | 0.391 |
| 1Ab - 0.5 | 0.326 | 0.208 |
| 1Ab - 0.25 | 0.186 | 0.108 |
| Buffer | 0.015 | 0.016 |
| Neg. Control | 0.019 | 0.011 |
| 3Bb1 - 16 in neg. seed | 0.017 | 0.009 |
| 3Bb1 - 4 in neg. seed | 0.014 | 0.008 |
| 3Bb1 - 1 in neg. seed | 0.014 | 0.010 |
| Neg. Seed | 0.014 | 0.007 |
| 1Ab - 8 in neg. seed | 1.241 | 1.007 |
| 1Ab - 2 in neg. seed | 0.473 | 0.378 |
| 1Ab - 0.5 in neg. seed | 0.171 | 0.139 |
| Neg. Seed | 0.019 | 0.009 |

### EXAMPLE 14

This example illustrates a multiplex ELISA for detecting BtCry1F (1F) and BtCry1Ab (1Ab) in a sample.

A mixture of polyclonal antibodies against 1F and 1Ab at a ratio of 2 to 1 was coated to the bottom and sides of the wells of ELISA plates using standard methods for coating ELISA plate wells with antibodies. The antibodies are available from Agdia, Inc., Elkhart, Ind.

The samples consisted of (1) 50% 1F and 1Ab positive seed (a equal mixture of 1F and 1Ab seeds) ground into powder and extracted in 1×PBST, (2) 25% 1F and 1Ab positive seed, (3) 12.5% 1F and 1Ab positive seed, (4) 6.3% 1F and 1Ab positive seed, (5) 3.1% 1F and 1Ab positive seed, (6) 1.6% 1F and 1Ab positive seed, (7) PBST, and (8) negative control. 1F and 1Ab are available from Agdia, Inc. Each sample was added to two wells of the above ELISA plate prepared as above with 100 μL sample per well. A mixture of monoclonal antibody specific for 1F and conjugated to alkaline phosphatase and polyclonal antibody specific for 1Ab and conjugated to peroxidase was added (100 μL/well) and the samples plus enzyme conjugates were incubated at room temperature for about 1 hour. The antibodies were at a 1:1 ratio in Ready to Use Buffer (RUB2). Afterwards, the samples and enzyme conjugate were removed from the wells ad the wells washed 6-7 times with 1×PBST. The wells were soaked about 3 minutes in 1×PBST and then the 1×PBST was decanted.

Next, a solution of Ready PNP was added to al wells (100 μL/well), and after 30-60 minutes at room temperature, the absorbance at 405 nm was read using an ELISA reader. After reading at 405 nm, the substrate was removed and the wells washed 4-5 times with 1×PBST. Then a solution of Prestained TMB was added (100 μL/well), and after 20 minutes at room temperature, the absorbance at 650 nm was read using an ELISA reader. Ready PNP and Prestained TMB are available from Kem-En-Tec Diagnostics, København, Denmark. The results are shown in Tables 33 and 34.

TABLE 33
Absorbance read at 405 nm

| 1F + 1Ab Seed | 1 | 2 | 3 | 4 |
|---|---|---|---|---|
| 50% | 1.403 | 1.529 | 1.579 | 1.877 |
| 25% | 1.186 | 1.274 | 1.411 | 1.496 |
| 12.5% | 0.637 | 0.663 | 0.776 | 0.876 |
| 6.3% | 0.363 | 0.365 | 0.408 | 0.512 |
| 3.1% | 0.205 | 0.204 | 0.242 | 0.277 |
| 1.6% | 0.105 | 0.113 | 0.131 | 0.172 |
| Buffer | 0.026 | 0.030 | 0.030 | 0.032 |
| Neg. Control | 0.026 | 0.030 | 0.028 | 0.029 |

TABLE 34
Absorbance read at 650 nm

| 1F + 1Ab Seed | 1 | 2 | 3 | 4 |
|---|---|---|---|---|
| 50% | 1.191 | 1.219 | 1.190 | 1.247 |
| 25% | 0.795 | 0.751 | 0.788 | 0.854 |
| 12.5% | 0.343 | 0.359 | 0.388 | 0.435 |
| 6.3% | 0.216 | 0.171 | 0.189 | 0.222 |
| 3.1% | 0.100 | 0.091 | 0.113 | 0.122 |
| 1.6% | 0.057 | 0.050 | 0.070 | 0.075 |
| Buffer | 0.009 | 0.009 | 0.021 | 0.021 |
| Neg. Control | 0.019 | 0.022 | 0.028 | 0.027 |

### EXAMPLE 15

This example illustrates a multiplex ELISA for detecting BtCry1F (1F) and BtCry1Ab (1Ab) in a sample.

A mixture of polyclonal antibodies against 1F and 1Ab at a ratio of 2 to 1 was coated to the bottom and sides of the wells of ELISA plates using standard methods for coating ELISA plate wells with antibodies. The antibodies are available from Agdia, Inc., Elkhart, Ind.

The samples consisted of (1) 50% 1F and 1Ab positive seed (an equal mixture of 1F and 1Ab seeds) ground into powder and extracted in 1×PBST, (2) 25% 1F and 1Ab positive seed, (3) 12.5% 1F and 1Ab positive seed, (4) 6.3% 1F and 1Ab positive seed, (5) 3.1% 1F and 1Ab positive seed, (6) 1.6% 1F and 1Ab positive seed, and (7) PBST. 1F and 1Ab are available from Agdia, Inc. Each sample was added to two wells of the above ELISA plate prepared as above with 100 μL sample per well. A mixture of monoclonal antibody specific for 1F and conjugated to alkaline phosphatase and polyclonal antibody specific for 1Ab and conjugated to peroxidase was added (100 μL/well) and the samples plus enzyme conjugates were incubated at room temperature for about two hours. The antibodies were at a 1:1 ratio in Ready to Use Buffer (RUB2). Afterwards, the samples and enzyme conjugate were removed from the wells and the wells washed 6-7 times with 1×PBST. The wells were soaked about 3 minutes in 1×PBST and then the 1×PBST was decanted.

Next, a solution of Ready PNP was added to all wells (100 μL/well), and after 30-60 minutes at room temperature, the absorbance at 405 nm was read using an ELISA reader. After reading at 405 nm, the substrate was removed and the wells washed 4-5 times with 1×PBST. Then a solution of Prestained TMB was added (100 μL/well), and after 20 minutes at room temperature, the absorbance at 650 nm was read using an ELISA reader. Ready PNP and Prestained TMB are available from Kem-En-Tec Diagnostics, København, Denmark. The results are shown in Tables 35 and 36.

TABLE 35
Absorbance read at 405 nm

| 1F + 1Ab Seed | 1 | 2 | 3 |
|---|---|---|---|
| 50% | 3.000 | 3.000 | 2.718 |
| 25% | 2.694 | 3.000 | 2.456 |
| 12.5% | 1.686 | 1.962 | 1.815 |
| 6.3% | 1.017 | 1.156 | 1.096 |
| 3.1% | 0.515 | 0.631 | 0.601 |
| 1.6% | 0.276 | 0.297 | 0.298 |
| Buffer | 0.041 | 0.061 | 0.029 |

TABLE 36
Absorbance read at 650 nm

| 1F + 1Ab Seed | 1 | 2 | 3 |
|---|---|---|---|
| 50% | 2.300 | 2.058 | 2.044 |
| 25% | 1.933 | 1.739 | 1.624 |
| 12.5% | 0.986 | 0.925 | 0.847 |
| 6.3% | 0.588 | 0.544 | 0.466 |
| 3.1% | 0.321 | 0.306 | 0.261 |
| 1.6% | 0.178 | 0.160 | 0.141 |
| Buffer | 0.059 | 0.051 | 0.038 |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. In a method for detecting an antigen in a sample by means of an enzyme linked immunoassay (ELISA) using an enzyme labeled conjugate so that the enzyme label is detected in the assay by reaction with a chromogenic substrate for the enzyme, the improvement which comprises:
sequentially determining the presence of at least two different antigens in a single assay by two different enzymatic reactions of at least two enzyme labeled conjugates with two different chromogenic substrates for the enzymes in the ELISA assay, wherein the antigen is immobilized on a solid support during the sequential enzymatic reactions in an indirect, direct or competitive assay and wherein the at least two different analytes are each detected without interference in the presence of the analytes, enzyme labeled conjugates and chromogenic substrates.

2. The method of claim 1 wherein the analyte is immobilized directly on the solid support.

3. The method of claim 1 wherein the analyte is immobilized indirectly on the solid surface by an antibody bound to the solid support.

4. The method of claims 1 or 3 wherein the assay is direct.

5. The method of claims 1 or 3 wherein the assay is indirect.

6. The method of claims 1 or 3 wherein the assay is competitive.

7. A method for detecting at least two different antigens in a single enzyme-linked immunosorbent assay (ELISA) which comprises:
(a) providing a solid support which is capable of directly binding the analytes;
(b) providing enzyme labeled antibodies which are capable of binding to each of the antigens bound to the solid support;
(c) contacting the antigens bound to the solid support with the enzyme labeled antibodies; and
(d) detecting whether the sample contains each of the analytes by sequentially adding a chromogenic substrate specific for each of the enzyme labeled antibodies to be detected to produce chromogens which are detected, wherein the at least two different analytes are each detected without interference in the presence of the analytes, enzyme labeled antibodies and chromogenic substrates.

8. A method for detecting at least two analyte species in a single enzyme linked immunosorbent assay which comprises:
(a) providing a solid support which is capable of directly binding analyte;
(b) providing a first antibody which selectively binds to each of the analytes;
(c) providing anti-first antibody second antibodies each labeled with a different enzyme;
(d) contacting each of the analytes bound to the support with the first antibodies to produce first complexes;
(e) contacting the first complexes with the second antibodies each labeled with the different enzymes to produce second complexes; and
(f) detecting whether the sample contains each of the second complexes by sequentially adding different chromogenic substrates specific for each of the enzyme labels of each of the second antibodies to be detected to produce chromogens, wherein the at least two different analytes are each detected without interference in the presence of the analytes, enzyme labeled antibodies and chromogenic substrates.

9. A method for detecting two or more analyte species in a single enzyme-linked immunosorbent assay (ELISA), which comprises:
(a) providing for each analyte species to be detected, an antibody specific for the analyte species immobilized on a solid support;
(b) contacting the antibodies immobilized on the solid support to a liquid sample suspected of containing at least one of the analyte species for a time sufficient for the antibodies to bind the analyte species;
(c) removing the solid support from the liquid sample and washing the solid support to remove unbound material;
(d) contacting the solid support to a solution comprising for each analyte species to be detected, an antibody specific for the analyte species to be detected conjugated to an enzyme label wherein the enzyme label for each antibody is different for a time sufficient for the antibodies to bind the analyte species bound by the immobilized antibodies;
(e) removing the solid support from the solution and washing the solid support to remove unbound antibodies; and
(f) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

10. The method of claim 9 wherein the solid support is a well of an ELISA plate.

11. The method of claim 9 wherein the chromogenic substrate is soluble and is converted to a soluble color.

12. The method of claim 9 wherein the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

13. The method of claim 9 wherein the enzyme label is selected from the group consisting of peroxidase, alkaline phosphatase, penicillinase, β-galactosidase, urease, and β-glucoronidase.

14. The method of claim 9 wherein the analyte species are from a plant pathogen.

15. The method of claim 9 wherein the analyte species are produced by a transgenic plant.

16. A method for detecting two analytes in a single enzyme-linked immunosorbent assay (ELISA), which comprises:
(a) providing a first antibody specific for a first analyte and a second antibody specific for a second analyte immobilized on a solid support;
(b) contacting the antibodies immobilized on the solid support to a liquid sample suspected of containing one or both of the analytes for a time sufficient for the antibodies to bind the analytes;
(c) removing the solid support from the liquid sample and washing the solid support to remove unbound material;
(d) contacting the solid support to a solution comprising a third antibody specific for the first analyte and a fourth antibody specific for the second analyte wherein the third antibody is conjugated to a first enzyme label and the fourth antibody is conjugated to a second enzyme label for a time sufficient for the third and fourth antibodies to bind the analytes bound by the first and second antibodies;
(e) removing the solid support from the solution and washing the solid support to remove unbound antibodies;
(f) adding a first chromogenic substrate for the first enzyme label wherein conversion of the first chromogenic substrate to a detectable color by the first enzyme label indicates that the sample contains the first analyte;
(g) removing the first chromogenic substrate; and
(h) adding a second chromogenic substrate for the second enzyme label wherein conversion of the second chromogenic substrate to a detectable color by the second enzyme label indicates that the sample contains the second analyte.

17. The method of claim 16 wherein the solid support is a well of an ELISA plate.

18. The method of claim 16 wherein the chromogenic substrate is soluble and is converted to a soluble color.

19. The method of claim 16 wherein the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

20. The method of claim 16 wherein the enzyme label is selected from the group consisting of peroxidase, alkaline phosphatase, penicillinase, β-galactosidase, urease, and β-glucoronidase.

21. The method of claim 16 wherein the analytes are from a plant pathogen.

22. The method of claim 16 wherein the analytes are produced by a transgenic plant.

23. A method for determining whether a plant material is derived from a transgenic plant which comprises one or more heterologous genes by detecting the products produced by the one or more heterologous genes, which comprises:
(a) providing a liquid sample from the plant material;
(b) providing a solid support having a mixture of antibodies immobilized thereon wherein the mixture comprises antibodies specific for the products produced by the one or more heterologous genes;
(c) contacting the antibodies immobilized on the solid support to the liquid sample for a time sufficient for the antibodies to bind the products;
(d) removing the solid support from the liquid sample and washing the solid support to remove unbound material;
(e) contacting the solid support to a solution comprising for each product to be detected, an antibody specific for the product to be detected conjugated to an enzyme label wherein the enzyme label for each antibody is different for a time sufficient for the antibodies to bind the products bound by the immobilized antibodies;
(f) removing the solid support from the solution and washing the solid support to remove unbound antibodies; and
(g) determining whether the sample contains each product by sequentially detecting the enzyme labels by adding a chromogenic substrate specific for the enzyme label to be detected wherein conversion of the chromogenic substrate to a detectable color indicates the sample contains the product.

24. The method of claim 23 wherein the solid support is a well of an ELISA plate.

25. The method of claim 23 wherein the chromogenic substrate is soluble and is converted to a soluble color.

26. The method of claim 23 wherein the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

27. The method of claim 23 wherein the enzyme label is selected from the group consisting of peroxidase, alkaline phosphatase, penicillinase, β-galactosidase, urease, and β-glucoronidase.

28. A kit for an ELISA comprising:
(a) a microtiter plate having a multiplicity of wells, each well having immobilized therein a mixture of two or more antibody species wherein each antibody species is specific for a particular analyte species;
(b) two or more first containers, each first container containing an antibody species conjugated to a particular enzyme label, wherein each antibody species is specific for the particular analyte species; and
(c) two or more second containers, each second container containing a chromogenic substrate, wherein each chromogenic substrate is specific for the particular enzyme label.

29. The kit of claim 28 wherein the kit comprises one first container which contains a mixture of the antibody species wherein each antibody species is specific for the particular analyte species and is conjugated to a particular enzyme label.

30. The kit of claim 28 wherein the chromogenic substrate is soluble and is converted to a soluble color.

31. The kit of claim 28 wherein the chromogenic substrate is selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

32. The kit of claim 28 wherein the enzyme label is selected from the group consisting of peroxidase, alkaline phosphatase, penicillinase, β-galactosidase, urease, and β-glucoronidase.

33. A method for detecting two or more analyte species in a single enzyme-linked immunosorbent assay (ELISA), which comprises:
(a) providing for each analyte species to be detected, an antibody specific for the analyte species immobilized on a solid support;
(b) providing a solution comprising for each analyte species to be detected, an antibody specific for the analyte species to be detected conjugated to an enzyme label wherein the enzyme label for each antibody is different;
(c) contacting a mixture comprising a sample suspected of containing at least one of the analyte species and the solution with the antibodies immobilized on the solid support for a time sufficient for the antibodies to bind the analyte species;
(d) removing the solid support from the mixture and washing the solid support to remove unbound antibodies; and
(e) determining whether the sample contains each analyte species by sequentially detecting the enzyme labels by adding a chromogenic substrate specific for the enzyme label to be detected wherein conversion of the chromogenic substrate to a detectable color indicates the sample contains the analyte species.

34. The method of claim 33 wherein the solid support is a well of an ELISA plate.

35. The method of claim 33 wherein the chromogenic substrate is soluble and is converted to a soluble color.

36. The method of claim 33 wherein the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

37. The method of claim 33 wherein the enzyme label is selected from the group consisting of peroxidase, alkaline phosphatase, penicillinase, β-galactosidase, urease, and β-glucoronidase.

38. The method of claim 33 wherein the analyte species are from a plant pathogen.

39. The method of claim 33 wherein the analyte species are produced by a transgenic plant.

40. A method for determining whether a plant material is derived from a transgenic plant which comprises one or more heterologous genes by detecting the products produced by the one or more heterologous genes, which comprises:
(a) providing a liquid sample from the plant material;
(b) providing a solid support having a mixture of antibodies immobilized thereon wherein the mixture comprises antibodies specific for the products produced by the one or more heterologous genes;
(c) providing a solution comprising for each product to be detected, an antibody specific for the product to be detected conjugated to an enzyme label wherein the enzyme label for each antibody is different for a time sufficient for the antibodies to bind the products bound by the immobilized antibodies to produce a mixture;
(d) contacting the antibodies immobilized on the solid support to a mixture comprising the sample and the solution for a time sufficient for the antibodies to bind the products;
(e) removing the solid support from the mixture and washing the solid support to remove unbound material; and
(f) determining whether the sample contains each product by sequentially detecting the enzyme labels by adding a chromogenic substrate specific for the enzyme label to be detected wherein conversion of the chromogenic substrate to a detectable color indicates the sample contains the product.

41. The method of claim 40 wherein the solid support is a well of an ELISA plate.

42. The method of claim 40 wherein the chromogenic substrate is soluble and is converted to a soluble color.

43. The method of claim 40 wherein the chromogenic substrates are selected from the group consisting of o-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), tetramethylbenzidine (TMB), bromothymol blue (BTB), bromochloroindolyl phosphate (BCP), bromocresol green (BCG), soluble two component 5-bromo-4-chloroindoxyl phosphate (BCIP) and 3-(4,5-dimethyl-2-thiazolyl)-2,5-diphenyl-2H-tetrazolium salt (MTT), pNPP or PNP (para-nitrophenyl phosphate), starch-iodine-penicillin V, bromothymol blue-penicillin V, and ONPG.

44. The method of claim 40 wherein the enzyme label is selected from the group consisting of peroxidase, alkaline phosphatase, penicillinase, β-galactosidase, urease, and β-glucoronidase.

45. The method of claim 9 wherein a purified analyte is complexed with a portion of the antibody in step (a) to provide a competitive direct assay for the analyte in the sample.

46. The method of claim 16 wherein the first and second antibodies of step (a) are complexed with purified analytes to provide a competitive indirect assay for the analyte in the sample.

47. The method of claim 9 wherein the analyte is a product of a heterologous gene in a plant.

48. The method of claim 16 wherein the analyte is a product of a heterologous gene in a plant.

49. A kit for an ELISA for two or more analytes in a single assay comprising:
(a) a solid support having immobilized therein a mixture of two different antibodies specific for the analytes or purified analytes, wherein each of the antibodies is specific for a particular of the analytes;
(b) one or more first containers each of the first containers containing second antibodies each labeled with different enzymes, which second antibodies are specific for each of the analytes; and
(c) two or more second containers, each of the two second containers containing a chromogenic substrate which is specific for each of the enzyme labels, wherein the analytes and antibodies are non-interfering in the assay.

50. The kit of claim 49 wherein in addition a purified analyte is bound to the solid support to provide for a competitive assay.

51. The kit of claim 49 wherein an antibody for the analyte is bound to the solid support.

52. The kit of claim 49 wherein the analyte is bound directly to the support.

53. A kit for an ELISA for two or more analytes in a single assay comprising:
(a) a solid support having immobilized thereon a mixture of two different first antibodies or a purified analyte, wherein each of the antibodies is specific for a particular of the analytes;
(b) one or more first containers containing second antibodies which antibodies are specific for each of the analytes;
(c) one or more second containers, each of the second containers containing anti-antibody second antibodies each conjugated to a different enzyme label which binds to the second antibodies; and
(d) two or more third containers containing a chromogenic substrate which is specific for each of the enzyme labels, wherein the analytes and antibodies are non-interfering in the assay.

54. The kit of claim 53 wherein a purified analyte is bound to the solid support to provide a competitive assay for the analyte.

55. The kit of claim 53 wherein an antibody is bound to the solid support.

56. The kit of claim 53 wherein the analyte is bound directly to the support.
